(19) United States
(12) Patent Application Publication
(10) Pub. No.: US 2026/0308301 A1
(43) Pub. Date: Oct. 8, 2026

(54) TERTIARY AMINO-AMIDE COMPOSITION USEFUL IN MAKING POLYURETHANE POLYMERS

(71) Applicant: EVONIK OPERATIONS GMBH, Essen (DE)

(72) Inventors: Juan Jesus BURDENIUC, Colmar, PA (US); Janine Dubbert, Haltern am See (DE); David Zugell, Pottstown, PA (US); Renee Jo Keller, Orwigsburg, PA (US); James Douglas Tobias, Center Valley, PA (US)

(73) Assignee: EVONIK OPERATIONS GMBH, Essen (DE)

(21) Appl. No.: 18/998,464

(22) PCT Filed: Jul. 13, 2023
(86) PCT No.: PCT/EP2023/069424

§ 371 Date: Jan. 25, 2025

Related U.S. Application Data
(60) Provisional application No. 63/393,103, filed on Jul. 28, 2022.

(51) Int. Cl. C08G 18/18; C07C 233/05; C08G 18/16

(52) U.S. Cl. CPC C08G 18/1833; C07C 233/05; C08G 18/163

(57) ABSTRACT

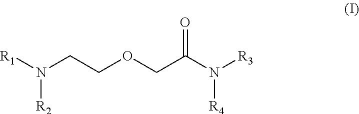

A catalyst composition comprising at least one compound represented by formula (I): wherein: R₁, R₂, and R₃, are each independently C₁-C₃ alkyl, or C₂-C₆ alkenyl linear or branched; and R₄ is hydrogen, C₁-C₃ alkyl, or C₂-C₆ alkenyl linear or branched.

### FIELD OF THE INVENTION

The field of invention is a composition and the use of new tertiary amino-amides as catalysts useful for the production of polyurethane foam.

### BACKGROUND OF THE INVENTION

Polyurethane foam compositions are typically prepared by reacting an isocyanate and a premix which consists of isocyanate-reactive components such as a polyol. The premix optionally also contains other components such as water, flame retardants, blowing agents, foam-stabilizing surfactants, and catalysts to promote the reactions of isocyanate with polyol to make urethane, with water to make CO₂ and urea, and with excess isocyanate to make isocyanurate (trimer).

The blowing agent in the premix is usually classified as chemical blowing agents or physical blowing agents. A chemical blowing agent is typically a substance that can produce gas when all the reactive components are mixed to produce a polyurethane foam. Examples of chemical blowing agents include water and formic acid. Water is the most common chemical blowing agent that can react with the isocyanate functionality to produce carbon dioxide. Water is commonly used in many types of polyurethane materials including rigid, semi-rigid and flexible polyurethane foam. Formic acid can also be used as blowing agent producing a mixture of carbon dioxide and carbon monoxide. Physical blowing agents on the other hand are liquids or gases with a boiling point sufficiently low to be vaporized by the heat released during the polymerization reaction.

Examples of blowing agents useful in the production of insulating polyurethane foam include but are not limited to hydrofluorocarbons, hydrofluoroolefins, hydrofluorochloroolefins, hydrochlorofluorocarbons, formates, ketones such as acetone and hydrocarbons such as pentane and cyclopentane.

Unlike simple hydrocarbons, such as pentane, halogen containing molecules such as chrolofluorocarbons (CFCs), hydrochlorofluorocarbons (HCFCs) and hydrofluorocarbons (HFCs) are far less flammable and safer to use in foam production. However, they either harm the ozone layer or contribute in other ways to global warming. In contrast, hydrofluoroolefins (HFOs) and hydrochlorofluoroolefins (HCFOs) are very efficient and environmentally friendly blowing agents with a much lower global warming potential (GWP) and zero ozone depleting potential (ODP).

The proper selection and combination of the components in the polyol premix and the isocyanate can be useful for the production of polyurethane foam that are either flexible, semi-flexible or rigid. Polyurethane foam materials of various characteristics can be useful in multiple applications including flexible molded foam useful in car interior applications, flexible slabstock foam useful in furniture, bedding, carpeting, transportation equipment interior, rigid-spray applied, poured in place, and used in applications such as refrigerators, freezers, hot water heaters, insulation panels, garage doors, entry doors, and other various applications where insulation is desired.

U.S. Pat. No. 3,816,339 discloses a polyurethane catalyst consisting of a mixture of N,N-dimethylcyclohexylamine and N-methyl-dicyclohexylamine that has unique characteristics. N,N-dimethylcyclohexylamine is a typical tertiary amine polyurethane catalyst used in large scale for the production of various types of polyurethane foam grade and in particular rigid closed cell polyurethane polymers.

U.S. Pat. No. 6,737,446 discloses a method for preparing polyurethane foams comprises reacting an organic polyisocyanate and a polyol in the presence of water as a blowing agent, a cell stabilizer, and an acid-blocked tertiary amino alkyl amide catalyst compn I (A=CH, N; R¹═H, II; R², R³═H, C₁₋₆ linear or branched alkyl; R⁴, R⁵=C₁₋₆ linear or branched alkyl, C₂₋₅ alkylene; R⁶═C₅₋₃₅ linear or branched alkyl, alkenyl, aryl; n=1-3).

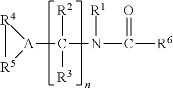

U.S. Pat. No. 7,169,823 discloses a method for preparing a polyurethane foam which comprises reacting an organic polyisocyanate and a polyol in the presence of water as a blowing agent, a cell stabilizer, and a tertiary amine amide catalyst composition represented by formula below wherein A, R¹-R⁶, and n are defined herein and wherein the tertiary amino amide catalyst of formula I is acid-blocked.

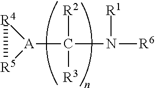

The ability of the tertiary amine catalyst to selectively promote either blowing or gelling is an important consideration in selecting a catalyst for preparing a particular polyurethane foam. If a catalyst promotes the blowing reaction too quickly, a substantial portion of the CO₂ will be evolved and will bubble out of the formulation before sufficient reaction of the isocyanate with the polyol has occurred, resulting in collapse of the foam and the production of a poor quality foam. On the other hand, if a catalyst promotes the gelling reaction too quickly, a substantial portion of the polymerization will have occurred before sufficient CO₂ has been evolved, resulting in insufficient blowing action and the production of a poor quality foam. Tertiary amine catalysts are generally malodorous and offensive and many are highly volatile due to their low molecular weight. The release of tertiary amine during foam processing may present significant safety and toxicity problems and the release of residual amine during customer handling is undesirable. On the other hand, low vapor pressure-high molecular weight amine catalysts are expected to require very high catalyst usage due to their low N/C ratio making the manufacturing cost very high.

Thus, there is a need for tertiary amine catalysts with low vapor pressures but relatively high N/C ratio so the catalytic activity can be maintained during the polymerization process while maintaining good physical and mechanical properties of the finished polymer.

### BRIEF SUMMARY OF THE INVENTION

The present invention relates to a method for preparing a polyurethane foam which comprises reacting an organic polyisocyanate and a polyol in the presence of polyurethane additives comprising blowing agent, a cell stabilizer, cross-linkers, and a catalyst composition comprising at least one compound represented by formula (I):

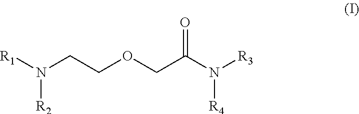

where R₁, R₂ and R₃ are a C₁-C₃ alkyl or C₂-C₆ alkenyl group linear or branched and R₄ is either hydrogen or C₁-C₆ alkyl or C₂-C₆ alkenyl group linear or branched. Preferably, in one embodiment R₁, R₂, and R₃ are each independently methyl groups and R₄ is hydrogen.

The instant invention can solve problems associated with conventional foam precursors by allowing the use of the inventive catalysts thereby improving and reducing the odor of the finished foam as well as providing good catalytic activity to yield polyurethane foam products with excellent physical properties.

The present invention provides a polyurethane catalyst and a polyol premix composition having the following benefits: a) provides a tertiary amine catalyst bearing amide functionality capable of providing good foam kinetics and cure including surface cure; b) improves the odor qualities as the amide does not participate in chain termination that results in detrimental foam physical properties; c) provides optimum catalytic activity and foam physical properties comparable with existing standards.

The catalyst composition is defined as at least one compound represented by formula (I):

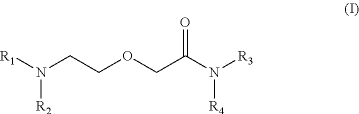

where R₁, R₂ and R₃ are a C₁-C₃ alkyl or C₂-C₆ alkenyl group linear or branched and R₄ is either hydrogen or C₁-C₆ alkyl or C₂-C₆ alkenyl group linear or branched. Preferably, in one embodiment R₁, R₂, and R₃ are each independently methyl groups and R₄ is hydrogen.

In an exemplary embodiment, a process includes providing a premix comprising at least one catalyst compound represented by formula (I) and contacting the pre-mix with at least one physical blowing agent including hydrofluorocarbons, hydrofluoroolefins, hydrofluorochloroolefins, hydrochlorofluorocarbons, formates, ketones such as acetone, hydrocarbons such as pentane and cyclopentane or chemical blowing agents such as water or formic acid.

In another exemplary embodiment, a polyurethane composition includes a polyol component, a catalyst composition, and an isocyanate component. The catalyst composition includes at least one compound represented by formula (I).

In another exemplary embodiment, a polyurethane product includes at least one catalyst compound represented by formula (I) and an isocyanate component.

In another exemplary embodiment, a catalyst composition includes at least one catalyst compound represented by formula (I) and/or a tertiary amine catalyst containing an isocyanate reactive group.

In another exemplary embodiment, a catalyst composition includes at least one catalyst compound represented by formula (I) and/or a tertiary amine catalyst containing no isocyanate reactive group.

Other features and advantages of the present invention will be apparent from the following more detailed description of the preferred embodiments, taken in conjunction with the accompanying drawings which illustrate, by way of example, the principles of the invention.

### Definitions

The following definitions are provided in order to aid those skilled in the art in understanding the detailed description of the present invention.
PUR—Polyurethane.
Isocyanate Index—The actual amount of polyisocyanate used divided by the theoretically required stoichiometric amount of polyisocyanate required to react with all the active hydrogen in the reaction mixture, multiplied by 100. Also known as (Eq NCO/Eq of active hydrogen)×100.
pphp—parts by weight per hundred weight parts polyol.
Polycat®-5—A commercial catalyst supplied by Evonik Corporation with a chemical name pentamethyldiethylenetriamine
Polycat®-8—A commercial catalyst supplied by Evonik Corporation with a chemical name dimethylaminocyclohexane

### DETAILED DESCRIPTION OF THE INVENTION

The present invention is directed to a catalyst composition comprising at least one compound represented by formula (I):

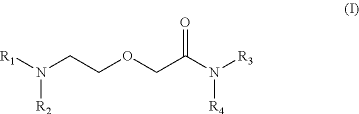

where R₁, R₂ and R₃ are a C₁-C₃ alkyl or C₂-C₆ alkenyl group linear or branched and R₄ is either hydrogen or C₁-C₆ alkyl or C₂-C₆ alkenyl group linear or branched. Preferably, in one embodiment R₁, R₂, and R₃ are each independently methyl groups and R₄ is hydrogen.

The present invention provides a polyurethane catalyst and a polyol premix composition having the following benefits: a) provides a tertiary amine catalyst bearing amide functionality capable of providing good foam kinetics and cure including surface cure; b) improves the odor qualities as the amide does not participate in chain termination that results in detrimental foam physical properties; c) provides optimum catalytic activity and foam physical properties comparable with existing standards.

Also, the present invention provides a method for preparing a polyurethane foam which comprises contacting at least one polyisocyanate with at least one active hydrogen-containing compound in the presence of at least one blowing agent and an effective amount of a catalyst composition as defined above in formula (I) in combination with a metal catalyst and/or a tertiary amine having or not an isocyanate reactive group.

Additionally, polyurethane foams can be produced with the novel catalyst system and novel compositions of the present invention by several methods known within the art.

Preferably, any amount of catalyst composition as defined in formula (I) above can be used in the compositions of the present invention.

The present invention discloses several types of ranges. These include, but are not limited to, a range of temperatures; a range of number of atoms; a range of foam density; a range of Isocyanate Index; and a range of pphp for the blowing agent, water, surfactant, flame retardant, and catalyst composition as defined in formula (I) above.

The present invention discloses a range of any type, which discloses individually each possible number that such a range could reasonably encompass, as well as any sub-ranges and combinations of sub-ranges encompassed therein. For example, when the present invention discloses a chemical moiety having a certain number of carbon atoms it will disclose individually every possible number that such a range could encompass.

For example, the disclosure that R¹ and R² are each independently C₁₋₃ alkyl linear or branched, C₂-C₆ alkenyl linear or branched mean for example that an alkyl group having up to 3 carbon atoms, or in alternative language a C₁₋₃ alkyl group, as used herein, refers to a “R¹” or “R²” group that can be selected independently from an alkyl group having 1, 2, or 3 carbon atoms, as well as a range between these two numbers for example, a C₂ to C₃ alkyl group.

Similarly, another representative example follows for the parts by weight of the catalyst composition as defined in formula (I) per hundred weight parts of the at least one active hydrogen-containing compound in a composition or a foam formulation. If the at least one active hydrogen-containing compound is an at least one polyol, the parts by weight per hundred weight parts polyol is abbreviated as pphp. Hence, by the disclosure that the catalyst composition as defined in formula (I) is present in an amount preferably from about 0.05 to about 10 pphp, for example, the pphp in the present invention can be selected from about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, or about 10. Likewise, all other ranges disclosed herein should be interpreted in a manner similar to these two examples.

Applicants reserve the right to proviso out or exclude any individual members of any such group, including any sub-ranges or combinations of sub-ranges within the group, that can be claimed according to a range or in any similar manner, if for any reason Applicants choose to claim less than the full measure of the disclosure, for example, to account for a reference that Applicants may be unaware of at the time of the filing of the application. Further, Applicants reserve the right to proviso out or exclude any individual substituents, analogs, compounds, ligands, structures, or groups thereof, or any members of a claimed group, if for any reason Applicants choose to claim less than the full measure of the disclosure, for example, to account for a reference that Applicants may be unaware of at the time of the filing of the application.

In another embodiment of the invention, the catalyst compositions can be used to make rigid foams (foam that is unable to bend or be forced out of shape) having a density of about 0.5 lb/ft³ to about 5 lb/ft³, about 1 lb/ft³ to about 4 lb/ft³ and in some cases about 2 lb/ft³ to about 3 lb/ft³. The catalyst compositions can be used to make close cell rigid foam such as those typically used in spray foam insulation and appliances having desirable physical properties including dimensional stability, adhesion, friability, thermal insulation and compression strength. In a further embodiment, the catalyst compositions can be used to make rigid foams having a density of about 0.5 lb/ft³ to about 5 lb/ft³, about 1 lb/ft³ to about 4 lb/ft³ and in some cases about 2 lb/ft³ to about 3 lb/ft³. Density can be measured in accordance with ASTM D3574 Test A.

In another embodiment of the invention, the catalyst compositions can be used to make flexible foam including flexible slabstock foam and flexible molded foam.

Flexible molded foams of the invention are characterized by excellent physical properties typically have target density (ASTM 3574-A) with range of about 28 to about 80 kg/m³, air flow (ASTM 3574-G) with range of about 40 to about 120 L/M, ILDs (indentation load deflection method ASTM 3574-B1) with range of about 150 to about 600 N, support factor (ASTM 3574-B1) with range of about 2.5 to about 3.5, preferably about 3, and resilience (ASTM 3574-H) range of about 40 to about 80%. In one embodiment of the invention a desirable foam has a Tensile/HA Tensile/Elongation/HA Elongation=DIN 53571—Range of about 80 to about 200%, a 50% Compression Set=ASTM D3574-D—Range of about 1 to about 20%, a HA Compression Set=ASTM D3574-J1 and J2—Range of about 5 to about 15%, and Tear=ASTM D3574-F—Range of about 150 to about 400.

In one embodiment of the invention, the catalyst composition as defined in formula (I) preferably comprises at least one member selected from the group consisting of 2-[2-(dimethylamino)ethoxy]-N-methyl-acetamide, 2-[2-(dimethylamino)ethoxy]-N,N-diethyl-acetamide, 2-[2-(dimethylamino)ethoxy]-N,N-dipropyl-acetamide, 2-[2-(dimethylamino)ethoxy]-N,N-dibutyl-acetamide, 2-[2-(dimethylamino)ethoxy]-N,N-dipentyl-acetamide, 2-[2-(dimethylamino)ethoxy]-N,N-dihexyl-acetamide, 2-[2-(dimethylamino)ethoxy]-N-methyl-N-ethyl-acetamide, 2-[2-(dimethylamino)ethoxy]-N-methyl-N-propyl-acetamide, 2-[2-(dimethylamino)ethoxy]-N-methyl-N-butyl-acetamide, 2-[2-(dimethylamino)ethoxy]-N-methyl-N-pentyl-acetamide, 2-[2-(dimethylamino)ethoxy]-N-methyl-N-hexyl-acetamide, 2-[2-(dimethylamino)ethoxy]-N-ethyl-acetamide, 2-[2-(dimethylamino)ethoxy]-N-propyl-acetamide, 2-[2-(dimethylamino)ethoxy]-N-butyl-acetamide, 2-[2-(dimethylamino)ethoxy]-N-pentyl-acetamide, 2-[2-(dimethylamino)ethoxy]-N-hexyl-acetamide, 2-[2-(diethylamino)ethoxy]-N,N-dimethyl-acetamide, 2-[2-(diethylamino)ethoxy]-N,N-diethyl-acetamide, 2-[2-(diethylamino)ethoxy]-N,N-dipropyl-acetamide, 2-[2-(diethylamino)ethoxy]-N,N-dibutyl-acetamide, 2-[2-(diethylamino)ethoxy]-N,N-dipentyl-acetamide, 2-[2-(diethylamino)ethoxy]-N,N-dihexyl-acetamide, 2-[2-(diethylamino)ethoxy]-N-methyl-N-ethyl-acetamide, 2-[2-(diethylamino)ethoxy]-N-methyl-N-propyl-acetamide, 2-[2-(diethylamino)ethoxy]-N-methyl-N-butyl-acetamide, 2-[2-(diethylamino)ethoxy]-N-methyl-N-pentyl-acetamide, 2-[2-(diethylamino)ethoxy]-N-methyl-N-hexyl-acetamide, 2-[2-(diethylamino)ethoxy]-N-methyl-acetamide, 2-[2-(diethylamino)ethoxy]-N-ethyl-acetamide, 2-[2-(diethylamino)ethoxy]-N-propyl-acetamide, 2-[2-(diethylamino)ethoxy]-N-butyl-acetamide, 2-[2-(diethylamino)ethoxy]-N-pentyl-acetamide, 2-[2-(diethylamino)ethoxy]-N-hexyl-acetamide, 2-[2-(N-methyl-N-ethyl-amino)ethoxy]-N,N-dimethyl-acetamide, 2-[2-(N-methyl-N-ethyl-amino)ethoxy]-N,N-diethyl-acetamide, 2-[2-(N-methyl-N-ethyl-amino)ethoxy]-N,N-dipropyl-acetamide, 2-[2-(N-methyl-N-ethyl-amino)ethoxy]-N,N-dibutyl-acetamide, 2-[2-(N-methyl-N-ethyl-amino)ethoxy]-N,N-dipentyl-acetamide, 2-[2-(N-methyl-N-ethyl-amino)ethoxy]-N,N-dihexyl-acetamide, 2-[2-(N-methyl-N-ethyl-amino)ethoxy]-N-methyl-N-ethyl-acetamide, 2-[2-(N-methyl-N-ethyl-amino)ethoxy]-N-methyl-N-propyl-acetamide, 2-[2-(N-methyl-N-ethyl-amino)ethoxy]-N-methyl-N-butyl-acetamide, 2-[2-(N-methyl-N-ethyl-amino)ethoxy]-N-methyl-N-pentyl-acetamide, 2-[2-(N-methyl-N-ethyl-amino)ethoxy]-N-methyl-N-hexyl-acetamide, 2-[2-(N-methyl-N-ethyl-amino)ethoxy]-N-methyl-acetamide, 2-[2-(N-methyl-N-ethyl-amino)ethoxy]-N-ethyl-acetamide, 2-[2-(N-methyl-N-ethyl-amino)ethoxy]-N-propyl-acetamide, 2-[2-(N-methyl-N-ethyl-amino)ethoxy]-N-butyl-acetamide, 2-[2-(N-methyl-N-ethyl-amino)ethoxy]-N-pentyl-acetamide, 2-[2-(N-methyl-N-ethyl-amino)ethoxy]-N-hexyl-acetamide, and the like. Such compounds can be employed individually or in any combination thereof.

In one embodiment, the catalyst composition as defined in formula (I) can be used as the sole catalyst or alternatively in combination with at least one tertiary amine catalyst. The tertiary amine catalyst can have at least one isocyanate reactive group or alternatively it can be a conventional tertiary amine catalyst having no-isocyanate reactive groups. Examples of isocyanate reactive groups comprise a primary hydroxyl group, a secondary hydroxyl group, a primary amine group, a secondary amine group, a urea group or an amide group. Examples of tertiary amine catalysts having an isocyanate reactive group preferably include, but are not limited to N, N-bis(3-dimethylaminopropyl)-N-isopropanolamine, N, N-dimethylaminoethyl-N′-methyl ethanolamine, N, N,N′-trimethylaminopropylethanolamine, N, N-dimethylethanolamine, N, N-diethylethanolamine, N, N-dimethyl-N′, N′-2-hydroxy (propyl)-1,3-propylenediamine, dimethylaminopropylamine, (N, N-dimethylaminoethoxy) ethanol, methyl-hydroxy-ethyl-piperazine, bis(N, N-dimethyl-3-aminopropyl)amine, N, N-dimethylaminopropyl urea, diethylaminopropyl urea, N,N′-bis(3-dimethylaminopropyl) urea, N,N′-bis(3-diethylaminopropyl) urea, bis(dimethylamino)-2-propanol, 6-dimethylamino-1-hexanol, N-(3-aminopropyl) imidazole, N-(2-hydroxypropyl) imidazole, and N-(2-hydroxyethyl) imidazole, N,N-bis(dimethylaminopropyl)-N-(3-aminopropyl)amine; N,N-bis(3-dimethylaminopropyl)-N-{3-[bis(2-hydroxypropyl)]propylamine}; N,N-bis(3-dimethylaminopropyl)-N-{3-[bis(2-hydroxyethyl)]propylamine}; N,N′-bis[bis-N″,N″-(3-dimethylaminopropyl)-N″-(3-aminopropyl)]urea; N,N-bis(3-dimethylaminopropyl)-N-(3-aminopropyl)]urea; N,N-bis(3-dimethylaminopropyl)-N-(bis(2-hydroxypropyl)-3-aminopropyl)]amine; N,N-bis(3-dimethylaminopropyl)-N—[N′, N′-bis(2-hydroxypropyl)-3-aminopropyl]amine; N,N-bis(3-dimethylaminopropyl)-N-[(2-hydroxypropyl)-3-aminopropyl]amine; 2-[N-(dimethylaminoethoxyethyl)-N-methylamino]ethanol, N, N-dimethylaminoethyl-N′-methyl-N′-ethanol, dimethylaminoethoxyethanol, N, N,N′-trimethyl-N′-3-aminopropyl-bis(aminoethyl) ether, or a combination thereof. The weight ratio of suitable tertiary amines to the inventive catalyst can range from about 0 to about 100, about 0.1 to about 50 and in some cases about 1 to about 10.

In one embodiment, the tertiary amine catalyst component is highly volatile and is not isocyanate-reactive. For example, in one embodiment, the tertiary amine catalyst component is a volatile gelling catalyst and preferably is or includes diazobicyclooctane (triethylenediamine), 1,8-diazabicycloundec-7-ene, tris(dimethylaminopropyl) amine, dimethylaminocyclohexylamine, bis(dimethylaminopropyl)-N-methylamine, or combinations thereof. Additionally or alternatively, in one embodiment, the tertiary amine catalyst component is or includes a volatile blowing catalyst and preferably is or includes bis(dimethylaminoethyl) ether, pentamethyldiethylenetriamine, hexamethyltriethylenetetramine, heptamethyltetraethylenepentamine and related compositions and higher permethylated polyamines. Additionally or alternatively, in another embodiment, the tertiary amine catalyst component preferably is or includes a blowing catalyst having an isocyanate reactive group such as 2-[N-(dimethylaminoethoxyethyl)-N-methylamino]ethanol and related structures, alkoxylated polyamines, imidazole-boron compositions, amino propyl-bis(amino-ethyl) ether compositions, or combinations thereof.

In another embodiment, the catalyst of the present invention can also preferably be acid blocked with an acid including carboxylic acids (alkyl, substituted alkyl, alkylene, aromatic, substituted aromatic) sulfonic acids or any other organic or inorganic acid. Examples of preferable carboxylic acids include mono-acids, di-acids or poly-acids with or without isocyanate reactive groups. Examples of preferable carboxylic acids include formic acid, acetic acid, propionic acid, butanoic acid, pentanoic acid, neopentanoic acid, hexanoic acid, 2-ethylhexyl carboxylic acid, neohexanoic acid, octanoic acid, neooctanoic acid, heptanoic acid, neoheptanoic acid, nonanoic acid, neononanoic acid, decanoic acid, neodecanoic acid, undecanoic acid, neoundecanoic acid, dodecanoic acid, neododecanoic acid, myristic acid, pentadecanoic acid, hexadecanoic acid, heptadecanoic acid, octadecanoic acid, benzoic acid, oxalic acid, malonic acid, succinic acid, glutaric acid, adipic acid, pimelic acid, suberic acid, azelaic acid, sebacic acid, glycolic acid, lactic acid, tartaric acid, citric acid, malic acid, salicylic acid and the like. An acid blocked catalyst can be obtained by known methods using conventional equipment.

In another embodiment, the tertiary amine catalyst component is preferably used in conjunction with a transition metal catalyst. For example, in one embodiment, the tertiary amine catalyst component is preferably used with an organotin compound, tin (II) carboxylate salts, bismuth (III) carboxylate salts, or combinations thereof. Examples of preferable transition metal catalysts such as organotin compounds or bismuth carboxylates can comprise at least one member selected from the group consisting of dibutyltin dilaurate, dimethyltin dilaurate, dimethyltin diacetate, dibutyltin diacetate, dimethyltin dilaurylmercaptide, dibutyltin dilaurylmercaptide, dimethyltin diisooctylmaleate, dibutyltin diisooctylmaleate, dimethyltin bi(2-ethylhexyl mercaptacetate), dibutyltin bi(2-ethylhexyl mercaptacetate), stannous octate, other suitable organotin catalysts, or a combination thereof. Other metals can also be included, such as, for example, bismuth (Bi). Suitable bismuth carboxylate salts preferably include salts of pentanoic acid, neopentanoic acid, hexanoic acid, 2-ethylhexyl carboxylic acid, neohexanoic acid, octanoic acid, neooctanoic acid, heptanoic acid, neoheptanoic acid, nonanoic acid, neononanoic acid, decanoic acid, neodecanoic acid, undecanoic acid, neoundecanoic acid, dodecanoic acid, neododecanoic acid, and other suitable carboxylic acids. Other salts of transition metals of lead (Pb), iron (Fe), zinc (Zn) with pentanoic acid, neopentanoic acid, hexanoic acid, 2-ethylhexyl carboxylic acid, octanoic acid, neooctanoic acid, neoheptanoic acid, neodecanoic acid, neoundecanoic acid, neododecanoic acid, and other suitable carboxylic acids may also be included.

The catalyst composition as defined in formula (I) can be produced, for example for the case of 2-[2-(dimethylamino)ethoxy]-N-methyl-acetamide by following this procedure: a fixed bed tubular reactor, equipped with a 10 cc quartz preheat bed, was charged with 8.8 g of a CuO/ZnO/Al₂O₃ catalyst sold under the name T-4581 material by Siid Chemie, with a typical composition of 61% CuO, 28% ZnO, and 10%. Al₂O₃. The reactor was pressurized with nitrogen to 20.7 bar (300 psig), and then vented to ambient. The reactor pressure was maintained by means of a backpressure controller. The nitrogen purge was repeated two additional cycles, followed by three hydrogen purges. The reactor was then fed hydrogen at 500 scc/m and 20.7 bar (300 psig). The reactor was heated, at 1° C./minute with a resistance heater, to 250° C. and held at that temperature for 4 hr to reduce the catalyst. The hydrogen flow, metered via a mass flow controller, was adjusted to provide a 4/1 molar ratio of hydrogen/dimethylaminoethoxyethanol (DMAEE). DMAEE was fed to the reactor under pressure, via a constant flow syringe pump. MMA was co-fed to the reactor under pressure, via a constant flow syringe pump at an MMA/DMAEE molar ratio of 2/1. Effluent from the reactor was analyzed by GC to give approximately 12% of 2-[2-(dimethylamino)ethoxy]-N-methyl-acetamide which was separated and purified by distillation.

In another embodiment, the catalyst system or compositions of the present invention can preferably further comprise other catalytic materials such as carboxylate salts in any amount. Preferably, the other catalytic materials are selected from alkali metal salts, alkaline earth metal salts, and quaternary ammonium carboxylate salts including, but are not limited to, potassium formate, potassium acetate, potassium propionate, potassium butanoate, potassium pentanoate, potassium hexanoate, potassium heptanoate, potassium octoate, potassium 2-ethylhexanoate, potassium decanoate, potassium butyrate, potassium isobutyrate, potassium nonante, potassium stearate, sodium octoate, lithium stearate, sodium caprioate, lithium octoate, 2-hydroxypropyltrimethylammonium octoate solution, and the like, or any combination thereof.

Preferably, the amount of the other catalytic materials and salts can range from about 0 pphp to about 20 pphp, about 0.1 pphp to about 15 pphp and in some cases about 0.5 pphp to about 10 pphp.

It is also within the scope of the catalyst composition of this invention to include mixtures or combinations of more than one catalyst compound as defined in formula (I). Additionally, in another embodiment the catalyst composition of the present invention can preferably also further comprise at least one urethane catalyst having no isocyanate reactive groups.

The term “contact product” is used herein to describe compositions wherein the components are contacted together in any order, in any manner, and for any length of time. For example, the components can be contacted by blending or mixing. Further, contacting of any component can occur in the presence or absence of any other component of the compositions or foam formulations described herein. Combining additional catalyst components can be done by any method known to one of skill in the art. For example, in one embodiment of the present invention, catalyst compositions can be prepared by combining or contacting the catalyst composition as defined in formula (I) with at least one tertiary amine having or not at least one isocyanate reactive group and optionally with an alkali metal carboxylate salt. This typically occurs in solution form.

While compositions and methods are described in terms of “comprising” various components or steps, the compositions and methods can also “consist essentially of” or “consist of” the various components or steps.

### Polyisocyanates

Polyisocyanates that are useful in the PIR/PUR foam formation process preferably include, but are not limited to, hexamethylene diisocyanate, isophorone diisocyanate, phenylene diisocyanate, toluene diisocyanate (TDI), diphenyl methane diisocyanate isomers (MDI), hydrated MDI and 1,5-naphthalene diisocyanate. For example, 2,4-TDI, 2,6-TDI, and mixtures thereof, can be readily employed in the present invention. Other suitable mixtures of diisocyanates include, but are not limited to, those known in the art as crude MDI, or PAPI, which contain 4,4′-diphenylmethane diisocyanate along with other isomeric and analogous higher polyisocyanates. In another embodiment of this invention, prepolymers of polyisocyanates comprising a partially pre-reacted mixture of polyisocyanates and polyether or polyester polyol are suitable. In still another embodiment, the polyisocyanate comprises MDI, or consists essentially of MDI or mixtures of MDI's.

The catalyst system, compositions, and methods of producing PIR/PUR foam of the present invention can be used to manufacture many types of foam. This catalyst system is useful, for example, in the formation of foam products for rigid and flame retardant applications, which usually require a high Isocyanate Index. As defined previously, Isocyanate Index is the actual amount of polyisocyanate used divided by the theoretically required stoichiometric amount of polyisocyanate required to react with all the active hydrogen in the reaction mixture, multiplied by 100. For purposes of the present invention, Isocyanate Index is represented by the equation: Isocyanate Index=(Eq NCO/Eq of active hydrogen)×100, wherein Eq NCO is the number of NCO functional groups in the polyisocyanate, and Eq of active hydrogen is the number of equivalent active hydrogen atoms.

Foam products which are produced with an Isocyanate Index from about 10 to about 800 are within the scope of this invention. In accordance with other embodiments of the present invention, the Isocyanate Index ranges from about 20 to about 700, from about 30 to about 650, from about 50 to about 600, or from about 70 to about 500.

### Polyols

Active hydrogen-containing compounds for use with the foregoing polyisocyanates in forming the polyisocyanurate/polyurethane foams of this invention can be any of those organic compounds having at least two hydroxyl groups such as, for example, polyols. Polyols that are typically used in PIR/PUR foam formation processes include polyalkylene ether and polyester polyols. The polyalkylene ether polyol includes the poly(alkyleneoxide) polymers such as poly(ethyleneoxide) and poly(propyleneoxide) polymers and copolymers with terminal hydroxyl groups derived from polyhydric compounds, including diols and triols, These preferably include, but are not limited to, ethylene glycol, propylene glycol, 1,3-butane diol, 1,4-butane diol, 1,6-hexane diol, neopentyl glycol, diethylene glycol, dipropylene glycol, pentaerythritol, glycerol, diglycerol, trimethylol propane, cyclohexane diol, and sugars such as sucrose and like low molecular weight polyols.

Amine polyether polyols can be used in the present invention. These can be prepared when an amine such as, for example, ethylenediamine, diethylenetriamine, tolylenediamine, diphenylmethanediamine, or triethanolamine is reacted with ethylene oxide or propylene oxide.

In another embodiment of the present invention, a single high molecular weight polyether polyol, or a mixture of high molecular weight polyether polyols, such as mixtures of different multifunctional materials and/or different molecular weight or different chemical composition materials can be used.

In yet another embodiment of the present invention, polyester polyols can be used, including those produced when a dicarboxylic acid is reacted with an excess of a diol. Non-limiting examples include adipic acid or phathalic acid or phthalic anhydride reacting with ethylene glycol or butanediol. Polyols useful in the present invention can be produced by reacting a lactone with an excess of a diol, for example, caprolactone reacted with propylene glycol. In a further embodiment, active hydrogen-containing compounds such as polyester polyols and polyether polyols, and combinations thereof, are useful in the present invention.

Preferably, the polyol can have an OH number of about 5 to about 600, about 100 to about 600 and in some cases about 50 to about 100 and a functionality of about 2 to about 8, about 3 to about 6 and in some cases about 4 to about 6.

Preferably, the amount of polyol can range from about 0 pphp to about 100 pphp about 10 pphp to about 90 pphp and in some cases about 20 pphp to about 80 pphp.

### Blowing Agents

In accordance with the compositions, foam formulations, and methods of producing PIR/PUR foam within the scope of the present invention, suitable blowing agents that can be used alone or in combination preferably include, but are not limited to, water, methylene chloride, acetone, hydrofluorocarbons (HFCs), hydrochlorocarbons (HCCs), hydrofluoroolefins (HFOs), chlorofluoroolefins (CFOs), hydrochloroolefins (HCOs), hydrofluorochloroolefins (HFCOs), hydrochlorofluorocarbons (HCFCs), chloroolefins, formates and hydrocarbons. Examples of HFCs include, but are not limited to, HFC-245fa, HFC-134a, and HFC-365; illustrative examples of HCFCs include, but are not limited to, HCFC-141b, HCFC-22, and HCFC-123. Exemplary hydrocarbons include, but are not limited to, n-pentane, iso-pentane, cyclopentane, and the like, or any combination thereof. In one embodiment of the present invention, the blowing agent or mixture of blowing agents comprises at least one hydrocarbon. In another embodiment, the blowing agent comprises n-pentane. Yet, in another embodiment of the present invention, the blowing agent consists essentially of n-pentane or mixtures of n-pentane with one or more blowing agents. Examples of hydrohaloolefin blowing agents are HFO-1234ze (trans-1,3,3,3-Tetrafluoroprop-1-ene), HFO-1234yf (2,3,3,3-Tetrafluoropropene) and HFCO-1233zd (1-Propene, 1-chloro-3,3,3-trifluoro), among other HFOs.

In one embodiment, the blowing agent component comprises a hydrohaloolefin, preferably comprising at least one of trans-HFO-1234ze and HFCO-1233zd, and optionally a hydrocarbon, fluorocarbon, chlorocarbon, fluorochlorocarbon, halogenated hydrocarbon, ether, fluorinated ether, ester, aldehyde, ketone, carbon dioxide generating material, or combinations thereof. The hydrohaloolefin preferably comprises at least one halooalkene such as a fluoroalkene or chloroalkene containing from 3 to 4 carbon atoms and at least one carbon-carbon double bond. Preferred hydrohaloolefins non-exclusively include trifluoropropenes, tetrafluoropropenes such as (HFO-1234), pentafluoropropenes such as (HFO-1225), chlorotrifluoropropenes such as (HFO-1233), chlorodifluoro propenes, chlorotrifluoropropenes, chlorotetrafluoropropenes, and combinations of these. Other preferred blowing agents comprise the tetrafluoropropene, pentafluoropropene, and chlorotrifluoropropene compounds in which the unsaturated terminal carbon has not more than one fluorine or chlorine substituent. Included are 1,3,3,3-tetrafluoropropene (HFO-1234ze); 1,1,3,3-tetrafluoropropene; 1,2,3,3,3-pentafluoropropene (HFO-1225ye); 1,1,1-trifluoropropene; 1,1,1,3,3-pentafluoropropene (HFO 1225zc); 1,1,1,3,3,3-hexafluorobut-2-ene, 1,1,2,3,3-pentafluoropropene (HFO-1225yc); 1,1,1,2,3-pentafluoropropene (HFO-1225yez); 1-chloro-3,3,3-trifluoropropene (HFCO-1233zd); 1,1,1,4,4,4-hexafluorobut-2-ene or combinations thereof, and any and all structural isomers, geometric isomers, or stereoisomers of each of these. Preferred optional blowing agents non-exclusively include water, formic acid, organic acids that produce carbon dioxide when they react with an isocyanate, hydrocarbons; ethers, halogenated ethers; pentafluorobutane; pentafluoropropane; hexafluoropropane; heptafluoropropane; trans-1,2 dichloro-ethylene; methyl formate; 1-chloro-1,2,2,2-tetrafluoroethane; 1,1-dichloro-1-fluoroethane; 1,1,1,2-tetrafluoroethane; 1,1,2,2-tetrafluoroethane; 1-chloro-1,1-difluoroethane; 1,1,1,3,3-pentafluorobutane; 1,1,1,2,3,3,3-heptafluoropropane; trichlorofluoromethane; dichlorodifluoromethane; 1,1,1,3,3,3-hexafluoropropane; 1,1,1,2,3,3-hexafluoropropane; difluoromethane; difluoroethane; 1,1,1,3,3-pentafluoropropane; 1,1-difluoroethane; isobutane; normal pentane; isopentane; cyclopentane, or combinations thereof. The blowing agent component is usually present in the polyol premix composition in an amount of from about 1 wt. % to about 30 wt. %, preferably from about 3 wt. % to about 25 wt. %, and more preferably from about 5 wt. % to about 25 wt. %, by weight of the polyol premix composition. When both a hydrohaloolefin and an optional blowing agent are present, the hydrohaloolefin component is usually present in the blowing agent component in an amount of from about 5 wt. % to about 90 wt. %, preferably from about 7 wt. % to about 80 wt. %, and more preferably from about 10 wt. % to about 70 wt. %, by weight of the blowing agent component; and the optional blowing agent is usually present in the blowing agent component in an amount of from about 95 wt. % to about 10 wt. %, preferably from about 93 wt. % to about 20 wt. %, and more preferably from about 90 wt. % to about 30 wt. %, by weight of the blowing agent component.

Due to the discovery that chlorofluorocarbons (CFCs) can deplete ozone in the stratosphere, this class of blowing agents is not desirable for use. A chlorofluorocarbon (CFC) is an alkane in which all hydrogen atoms are substituted with chlorine and fluorine atoms. Examples of CFCs include trichlorofluoromethane and dichlorodifluoromethane.

The amount of blowing agent used can vary based on, for example, the intended use and application of the foam product and the desired foam stiffness and density. In the compositions, foam formulations and methods for preparing a polyisocyanurate/polyurethane foam of the present invention, the blowing agent is present in amounts from about 5 to about 80 parts by weight per hundred weight parts of the at least one active hydrogen-containing compound. In another embodiment, the blowing agent is present in amounts from about 10 to about 60, from about 15 to about 50, or from about 20 to about 40, parts by weight per hundred weight parts of the at least one active hydrogen-containing compound. If the at least one active hydrogen-containing compound is an at least one polyol, the blowing agent is present in amounts from about 5 to about 80 parts by weight per hundred weight parts polyol (pphp), from about 10 to about 60 pphp, from about 15 to about 50 pphp, or from about 20 to about 40 pphp.

If water is present in the formulation, for use as a blowing agent or otherwise, water is present in amounts up to about 60 parts by weight per hundred weight parts of the at least one active hydrogen-containing compound. Likewise, if the at least one active hydrogen-containing compound is an at least one polyol, water can range from 0 to about 15 pphp. In another embodiment, water can range from 0 to about 10 pphp, from 0 to about 8 pphp, from 0 to about 6 pphp, or from 0 to about 4 pphp.

### Urethane Catalyst

In one embodiment, conventional urethane catalysts having no isocyanate reactive group can preferably be employed to accelerate the reaction to form polyurethanes, and can be used as a further component of the catalyst systems and compositions of the present invention to produce polyisocyanurate/polyurethane foam. Urethane catalysts suitable for use herein preferably include, but are not limited to, metal salt catalysts, such as organotins, and amine compounds, such as triethylenediamine (TEDA), N-methylimidazole, 1,2-dimethyl-imidazole, N-methylmorpholine (commercially available as the DABCO® NMM catalyst), N-ethylmorpholine (commercially available as the DABCO® NEM catalyst), triethylamine (commercially available as the DABCO® TETN catalyst), N,N′-dimethylpiperazine, 1,3,5-tris(dimethylaminopropyl) hexahydrotriazine (commercially available as the Polycat® 41 catalyst), 2,4,6-tris(dimethylaminomethyl) phenol (commercially available as the DABCO TMR® 30 catalyst), N-methyldicyclohexylamine (commercially available as the Polycat® 12 catalyst), pentamethyldipropylene triamine (commercially available as the Polycat® 77 catalyst), N-methyl-N′-(2-dimethylamino)-ethyl-piperazine, tributylamine, pentamethyl-diethylenetriamine (commercially available as the Polycat® 5 catalyst), hexamethyl-triethylenetetramine, heptamethyltetraethylenepentamine, dimethylaminocyclohexyl-amine (commercially available as the Polycat® 8 catalyst), pentamethyldipropylene-triamine, triethanolamine, dimethylethanolamine, bis(dimethylaminoethyl) ether (commercially available as the DABCO® BL19 catalyst), tris(3-dimethylamino)-propylamine (commercially available as the Polycat® 9 catalyst), 1,8-diazabicyclo[5.4.0]undecene (commercially available as the DABCO® DBU catalyst) or its acid blocked derivatives, and the like, as well as any mixture thereof.

In another embodiment, the present invention can be used with tertiary amine catalysts having isocyanate reactive groups. Preferably, the isocyanate reactive groups present in the tertiary amine gelling co-catalyst consist essentially of primary amine, secondary amine, secondary-hydroxyl group, amide and urea. Examples of gelling catalysts preferably include N,N-bis(3-dimethylamino-propyl)-N-(2-hydroxypropyl)amine; N,N-dimethyl-N′,N′-bis(2-hydroxypropyl)-1,3-propylenediamine;

dimethylaminopropylamine (DMAPA); N-methyl-N-2-hydroxypropyl-piperazine, bis(dimethylaminopropyl)amine (POLYCAT® 15), dimethylaminopropylurea and N,N′-bis(3-dimethylaminopropyl) urea (DABCO® NE1060, DABCO® NE1070, DABCO® NE1080 and DABCO® NE1082), 1,3-bis(dimethylamino)-2-propanol, 6-dimethylamino-1-hexanol, N-(3-aminopropyl) imidazole, N-(2-hydroxypropyl) imidazole, N,N′-bis(2-hydroxypropyl) piperazine, N-(2-hydroxypropyl)-morpholine, N-(2-hydroxyethylimidazole); N,N-bis(3-dimethylaminopropyl)-N-{3-[bis(2-hydroxypropyl)]propylamine}; N,N-bis(3-dimethylaminopropyl)-N-{3-[bis(2-hydroxyethyl)]propylamine}; N,N-bis(dimethylaminopropyl)-N-(3-aminopropyl)-amine; N,N′-bis[bis-N″,N″-(3-dimethylaminopropyl)-N″-(3-aminopropyl)]urea; N,N-bis(3-dimethylaminopropyl)-N-(3-aminopropyl)]urea; N,N-bis(3-dimethylaminopropyl)-N-(bis(2-hydroxypropyl)-3-aminopropyl)]amine; N,N-bis(3-dimethylaminopropyl)-N—[N′,N′-bis(2-hydroxypropyl)-3-aminopropyl]amine; and N,N-bis(3-dimethylaminopropyl)-N-[(2-hydroxypropyl)-3-aminopropyl]amine. Examples of blowing co-catalysts containing isocyanate reactive groups that can be used with the above mentioned gelling catalysts preferably include 2-[N-(dimethylaminoethoxyethyl)-N-methylamino]ethanol (DABCO® NE200), N,N,N′-trimethyl-N′-3-aminopropyl-bis(aminoethyl) ether (DABCO® NE300).

Suitable urethane catalysts that can be used in combination with the catalyst in the present invention preferably also include acid blocked tertiary amines with acids including carboxylic acids (alkyl, substituted alkyl, alkylene, aromatic, substituted aromatic) sulfonic acids or any other organic or inorganic acid. Examples of carboxylic acids preferably include mono-acids, di-acids or poly-acids with or without isocyanate reactive groups. Examples of preferable carboxylic acids include formic acid, acetic acid, propionic acid, butanoic acid, pentanoic acid, neopentanoic acid, hexanoic acid, 2-ethylhexyl carboxylic acid, neohexanoic acid, octanoic acid, neooctanoic acid, heptanoic acid, neoheptanoic acid, nonanoic acid, neononanoic acid, decanoic acid, neodecanoic acid, undecanoic acid, neoundecanoic acid, dodecanoic acid, neododecanoic acid, myristic acid, pentadecanoic acid, hexadecanoic acid, heptadecanoic acid, octadecanoic acid, benzoic acid, oxalic acid, malonic acid, succinic acid, glutaric acid, adipic acid, pimelic acid, suberic acid, azelaic acid, sebacic acid, glycolic acid, lactic acid, tartaric acid, citric acid, malic acid, salicylic acid and the like. An acid blocked catalyst can be obtained by known methods using conventional equipment.

In another embodiment, the tertiary amine catalyst component can preferably also be used in conjunction with a metal catalyst. For example, in one embodiment, the tertiary amine catalyst component is preferably used with an organotin compound, tin (II) carboxylate salts, bismuth (III) carboxylate salts, or combinations thereof. Preferred examples of metal catalysts such as organotin compounds or bismuth carboxylates can comprise at least one member selected from the group consisting of dibutyltin dilaurate, dimethyltin dilaurate, dimethyltin diacetate, dibutyltin diacetate, dimethyltin dilaurylmercaptide, dibutyltin dilaurylmercaptide, dimethyltin diisooctylmaleate, dibutyltin diisooctylmaleate, dimethyltin bi(2-ethylhexyl mercaptacetate), dibutyltin bi(2-ethylhexyl mercaptacetate), stannous octoate, other suitable organotin catalysts, or a combination thereof. Other metals can also be included, such as, for example, bismuth (Bi). Suitable bismuth carboxylate salts preferably include salts of pentanoic acid, neopentanoic acid, hexanoic acid, 2-ethylhexyl carboxylic acid, neohexanoic acid, octanoic acid, neooctanoic acid, heptanoic acid, neoheptanoic acid, nonanoic acid, neononanoic acid, decanoic acid, neodecanoic acid, undecanoic acid, neoundecanoic acid, dodecanoic acid, neododecanoic acid, and other suitable carboxylic acids. Other salts of metals of lead (Pb), iron (Fe), zinc (Zn) with pentanoic acid, neopentanoic acid, hexanoic acid, 2-ethylhexyl carboxylic acid, octanoic acid, neooctanoic acid, neoheptanoic acid, neodecanoic acid, neoundecanoic acid, neododecanoic acid, and other suitable carboxylic acids may also be included.

In another embodiment, the present invention can preferably further comprise other catalytic materials such as carboxylate salts in any amount. Preferable examples of alkali metal, alkaline earth metal, and quaternary ammonium carboxylate salts include, but are not limited to, potassium formate, potassium acetate, potassium propionate, potassium butanoate, potassium pentanoate, potassium hexanoate, potassium heptanoate, potassium octoate, potassium 2-ethylhexanoate, potassium decanoate, potassium butyrate, potassium isobutyrate, potassium nonante, potassium stearate, sodium octoate, lithium stearate, sodium caprioate, lithium octoate, 2-hydroxypropyltrimethylammonium octoate solution, tetramethylammonium carboxylates, tetralkylammonium carboxylates such as tetramethylammonium pivalate (supplied by Evonik Corporation as DABCO® TMR7) and the like, or any combination thereof.

For preparing a polyisocyanurate/polyurethane foam of the present invention, the urethane catalyst can be present in the formulation from 0 to about 10 pphp, from 0 to about 8 pphp, from 0 to about 6 pphp, from 0 to about 4 pphp, from 0 to about 2 pphp, or from 0 to about 1 pphp. In another embodiment, the urethane catalyst is present from 0 to about 0.8 pphp, from 0 to about 0.6 pphp, from 0 to about 0.4 pphp, or from 0 to about 0.2 pphp.

### Miscellaneous Additives

Depending on the requirements during foam manufacturing or for the end-use application of the foam product, various additives can preferably be employed in the PIR/PUR foam formulation to tailor specific properties. These additives preferably include, but are not limited to, cell stabilizers, flame retardants, chain extenders, epoxy resins, acrylic resins, fillers, pigments, or any combination thereof. It is understood that other mixtures or materials that are known in the art can be included in the foam formulations and are within the scope of the present invention.

Cell stabilizers include surfactants such as organopolysiloxanes. Silicon surfactants can be present in the foam formulation in amounts from about 0.5 to about 10 pphp, about 0.6 to about 9 pphp, about 0.7 to about 8 pphp, about 0.8 to about 7 pphp, about 0.9 to about 6 pphp, about 1 to about 5 pphp, or about 1.1 to about 4 pphp. Useful flame retardants include halogenated organophosphorous compounds and non-halogenated compounds. A non-limiting example of a halogenated flame retardant is trichloropropylphosphate (TCPP). For example, triethylphosphate ester (TEP) and DMMP are non-halogenated flame retardants. Depending on the end-use foam application, flame retardants can be present in the foam formulation in amounts from 0 to about 50 pphp, from 0 to about 40 pphp, from 0 to about 30 pphp, or from 0 to about 20 pphp. In another embodiment, the flame retardant is present from 0 to about 15 pphp, 0 to about 10 pphp, 0 to about 7 pphp, or 0 to about 5 pphp. Chain extenders such as ethylene glycol and butane diol can also be employed in the present invention. Ethylene glycol, for instance, can also be present in the formulation as a diluent or solvent for the carboxylate salt catalysts of the present invention.

### Polyurethane Foam Formulation and Process

One preferred embodiment of the present invention provides for a polyurethane composition comprising the contact product of at least one active hydrogen-containing compound, at least one blowing agent, and at least one catalyst composition as defined above in formula (I).

Another preferred embodiment provides a composition comprising the contact product of at least one polyisocyanate, at least one blowing agent, and at least one catalyst composition as defined above in formula (I) used in combination with at least one tertiary amine having at least one isocyanate reactive group.

Another preferred embodiment provides a composition comprising the contact product of at least one polyisocyanate, at least one blowing agent, and at least one catalyst composition as defined above in formula (I) used in combination with at least one tertiary amine having no isocyanate reactive group.

In another preferred embodiment, the composition can further comprise the catalyst composition as defined above in formula (I) with at least one urethane catalyst having no isocyanate reactive group and at least one urethane catalyst having an isocyanate reactive group. Likewise, the compositions can preferably further comprise at least one additive selected from at least one cell stabilizer, at least one flame retardant, at least one chain extender, at least one epoxy resin, at least one acrylic resin, at least one filler, at least one pigment, or any combination thereof.

In another preferred embodiment, the present invention provides a method for preparing a polyurethane foam as well as a polyisocyanurate/polyurethane (PIR/PUR) foam which comprises contacting at least one polyisocyanate with at least one active hydrogen-containing compound, in the presence of at least one blowing agent and an effective amount of catalyst composition as defined above in formula (I). In accordance with the method of the present invention, PUR as well as PIR/PUR foams can be produced having a density from about 8 Kg/m³ to about 250 Kg/m³ (about 0.5 lb/ft³ to about 15.5 lb/ft³), or from about 24 Kg/m³ to about 60 Kg/m³ (about 1.5 lb/ft³ to about 3.75 lb/ft³).

Another preferred embodiment provides a method for preparing a polyurethane foam which comprises contacting at least one polyisocyanate with at least one active hydrogen-containing compound in the presence of at least one blowing agent and an effective amount of catalyst composition as defined above in formula (I), wherein the catalyst composition is present in a tertiary amine having or not an isocyanate reactive group.

Another preferred embodiment provides a method for preparing a polyurethane foam which comprises contacting at least one polyisocyanate with at least one active hydrogen-containing compound in the presence of at least one blowing agent and an effective amount of catalyst composition as defined above in formula (I), wherein the catalyst composition is present in combination with a metal catalyst, a tertiary amine having or not an isocyanate reactive group, or a combination thereof.

The present invention can be used in a wide range of methods for making rigid closed-cell foams, rigid open cell foams, flexible foam including flexible slabstock foam and flexible molded foam as well as semi-flexible foam and microcellular foam. Examples of suitable methods comprise pouring, molding, spraying, among other rigid foam production methods. In one embodiment the inventive method relates to a method for making a laminated foam. The inventive foam can be laminated to a wide range of substrates including wood, steel, paper and plastic.

The method for preparing PUR as well as PIR/PUR foams also can provide lower ammoniacal odor polyol premix when compared to other commercially available catalyst systems.

The catalyst composition as defined above in formula (I) is preferably present in the foam formulation in a catalytically effective amount. In PUR as well as in PIR/PUR foam formulations of the present invention, the catalyst composition is present in amounts from about 0.05 to about 20 parts by weight per hundred weight parts of the at least one active hydrogen-containing compound, excluding the weight contribution of the catalyst system diluent. In another embodiment, the catalyst composition is present in amounts from about 0.4 to about 10 parts by weight per hundred weight parts of the at least one active hydrogen-containing compound, or from about 0.8 to about 8 parts by weight per hundred weight parts of the at least one active hydrogen-containing compound. If the at least one active hydrogen-containing compound is an at least one polyol, the catalyst composition is present in amounts from about 0.05 to about 10 parts by weight per hundred weight parts polyol (pphp). In another embodiment, the catalyst composition is present in amounts from about 0.2 to about 9.5 pphp, about 0.4 to about 9 pphp, about 0.6 to about 8.5 pphp, or about 0.8 to about 8 pphp.

In accordance with one embodiment of the method of the present invention, the components of the foam formulation are contacted substantially contemporaneously. For example, at least one polyisocyanate, at least one active hydrogen-containing compound, at least one blowing agent and an effective amount of catalyst composition as defined above in formula (I), are contacted together. Given the number of components involved in PUR and PIR/PUR formulations, there are many different orders of combining the components, and one of skill in the art would realize that varying the order of addition of the components falls within the scope of the present invention. As well, for each of the different orders of combining the aforementioned components of the foam formulation, the foam formulation of the present invention can further comprise at least one urethane catalyst. In addition, the method of producing PIR/PUR foams can preferably further comprise the presence of at least one additive selected from at least one cell stabilizer, at least one flame retardant, at least one chain extender, at least one epoxy resin, at least one acrylic resin, at least one filler, at least one pigment, or any combination thereof. In one embodiment of the present invention, all of the components, including optional components, are contacted substantially contemporaneously.

In another embodiment of the present invention, a premix of ingredients other than the at least one polyisocyanate are contacted first, followed by the addition of the at least one polyisocyanate. For example, the at least one active hydrogen-containing compound, the at least one blowing agent, the at least one cell stabilizer, and the catalyst composition of the present invention are contacted initially to form a premix. The premix is then contacted with the at least one polyisocyanate to produce PUR or PIR/PUR foams in accordance with the method of the present invention. In a further embodiment of the present invention, the same method can be employed, wherein the premix preferably further comprises at least one urethane catalyst. Likewise, the premix can preferably further comprise at least one additive selected from at least one cell stabilizer, at least one flame retardant, at least one chain extender, at least one epoxy resin, at least one acrylic resin, at least one filler, at least one pigment, or any combination thereof.

One embodiment of the present invention provides a method for preparing a polyurethane, polyisocyanurate, polyisocyanurate/polyurethane foam comprising:
(a) forming a premix comprising:
i) at least one polyol;
ii) about 1 to about 80 parts by weight per hundred weight parts of the polyol (pphp) blowing agent;
iii) about 0.5 to about 10 pphp silicon surfactant;
iv) zero to about 60 pphp water;
v) zero to about 50 pphp flame retardant;
vi) zero to about 10 pphp urethane catalyst; and
vii) about 0.05 to about 20 pphp of a catalyst composition as defined above in formula (I); and
(b) contacting the premix with at least one polyisocyanate at an Isocyanate Index from about 10 to about 800.

The following is a list of preferred items of the invention:

Item 1. A catalyst composition comprising at least one compound represented by formula (I):

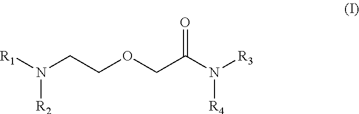

wherein:
R₁, R₂, and R₃, are each independently C₁-C₃ alkyl, or C₂-C₆ alkenyl linear or branched; and
R₄ is hydrogen, C₁-C₃ alkyl, or C₂-C₆ alkenyl linear or branched.

Item 2. The catalyst composition of item 1, wherein R₁, R₂, and R₃ are each independently methyl groups and R₄ is hydrogen.

Item 3. The catalyst composition of item 1, wherein the at least one compound represented by formula (I) is selected from the group consisting of 2-[2-(dimethylamino)ethoxy]-N-methyl-acetamide, 2-[2-(dimethylamino)ethoxy]-N,N-diethyl-acetamide, 2-[2-(dimethylamino)ethoxy]-N,N-dipropyl-acetamide, 2-[2-(dimethylamino)ethoxy]-N,N-dibutyl-acetamide, 2-[2-(dimethylamino)ethoxy]-N,N-dipentyl-acetamide, 2-[2-(dimethylamino)ethoxy]-N,N-dihexyl-acetamide, 2-[2-(dimethylamino)ethoxy]-N-methyl-N-ethyl-acetamide, 2-[2-(dimethylamino)ethoxy]-N-methyl-N-propyl-acetamide, 2-[2-(dimethylamino)ethoxy]-N-methyl-N-butyl-acetamide, 2-[2-(dimethylamino)ethoxy]-N-methyl-N-pentyl-acetamide, 2-[2-(dimethylamino)ethoxy]-N-methyl-N-hexyl-acetamide, 2-[2-(dimethylamino)ethoxy]-N-ethyl-acetamide, 2-[2-(dimethylamino)ethoxy]-N-propyl-acetamide, 2-[2-(dimethylamino)ethoxy]-N-butyl-acetamide, 2-[2-(dimethylamino)ethoxy]-N-pentyl-acetamide, 2-[2-(dimethylamino)ethoxy]-N-hexyl-acetamide, 2-[2-(diethylamino)ethoxy]-N,N-dimethyl-acetamide, 2-[2-(diethylamino)ethoxy]-N,N-diethyl-acetamide, 2-[2-(diethylamino)ethoxy]-N,N-dipropyl-acetamide, 2-[2-(diethylamino)ethoxy]-N,N-dibutyl-acetamide, 2-[2-(diethylamino)ethoxy]-N,N-dipentyl-acetamide, 2-[2-(diethylamino)ethoxy]-N,N-dihexyl-acetamide, 2-[2-(diethylamino)ethoxy]-N-methyl-N-ethyl-acetamide, 2-[2-(diethylamino)ethoxy]-N-methyl-N-propyl-acetamide, 2-[2-(diethylamino)ethoxy]-N-methyl-N-butyl-acetamide, 2-[2-(diethylamino)ethoxy]-N-methyl-N-pentyl-acetamide, 2-[2-(diethylamino)ethoxy]-N-methyl-N-hexyl-acetamide, 2-[2-(diethylamino)ethoxy]-N-methyl-acetamide, 2-[2-(diethylamino)ethoxy]-N-ethyl-acetamide, 2-[2-(diethylamino)ethoxy]-N-propyl-acetamide, 2-[2-(diethylamino)ethoxy]-N-butyl-acetamide, 2-[2-(diethylamino)ethoxy]-N-pentyl-acetamide, 2-[2-(diethylamino)ethoxy]-N-hexyl-acetamide, 2-[2-(N-methyl-N-ethyl-amino)ethoxy]-N,N-dimethyl-acetamide, 2-[2-(N-methyl-N-ethyl-amino)ethoxy]-N,N-diethyl-acetamide, 2-[2-(N-methyl-N-ethyl-amino)ethoxy]-N,N-dipropyl-acetamide, 2-[2-(N-methyl-N-ethyl-amino)ethoxy]-N,N-dibutyl-acetamide, 2-[2-(N-methyl-N-ethyl-amino)ethoxy]-N,N-dipentyl-acetamide, 2-[2-(N-methyl-N-ethyl-amino)ethoxy]-N,N-dihexyl-acetamide, 2-[2-(N-methyl-N-ethyl-amino)ethoxy]-N-methyl-N-ethyl-acetamide, 2-[2-(N-methyl-N-ethyl-amino)ethoxy]-N-methyl-N-propyl-acetamide, 2-[2-(N-methyl-N-ethyl-amino)ethoxy]-N-methyl-N-butyl-acetamide, 2-[2-(N-methyl-N-ethyl-amino)ethoxy]-N-methyl-N-pentyl-acetamide, 2-[2-(N-methyl-N-ethyl-amino)ethoxy]-N-methyl-N-hexyl-acetamide, 2-[2-(N-methyl-N-ethyl-amino)ethoxy]-N-methyl-acetamide, 2-[2-(N-methyl-N-ethyl-amino)ethoxy]-N-ethyl-acetamide, 2-[2-(N-methyl-N-ethyl-amino)ethoxy]-N-propyl-acetamide, 2-[2-(N-methyl-N-ethyl-amino)ethoxy]-N-butyl-acetamide, 2-[2-(N-methyl-N-ethyl-amino)ethoxy]-N-pentyl-acetamide, and 2-[2-(N-methyl-N-ethyl-amino)ethoxy]-N-hexyl-acetamide.

Item 4. The catalyst composition of any of items 1-3 further comprising a tertiary amine catalyst having or not an isocyanate reactive group.

Item 5. The catalyst composition of item 4, wherein the tertiary amine catalyst has at least one isocyanate reactive group comprising a primary hydroxyl group, a secondary hydroxyl group, a primary amine group, a secondary amine group, a urea group or an amide group.

Item 6. The catalyst composition of item 4 or 5, wherein the tertiary amine catalyst is selected from the group consisting of N, N-bis(3-dimethylaminopropyl)-N-isopropanolamine; N, N-dimethylaminoethyl-N′-methyl ethanolamine; N, N,N′-trimethylaminopropylethanolamine; N, N-dimethylethanolamine; N, N-diethylethanolamine; N, N-dimethyl-N′, N′-2-hydroxy (propyl)-1,3-propylenediamine; dimethylaminopropylamine; (N, N-dimethylaminoethoxy) ethanol; methyl-hydroxy-ethyl-piperazine; bis(N, N-dimethyl-3-aminopropyl)amine; N, N-dimethylaminopropyl urea;

diethylaminopropyl urea; N,N′-bis(3-dimethylaminopropyl) urea; N,N′-bis(3-diethylaminopropyl) urea; bis(dimethylamino)-2-propanol; 6-dimethylamino-1-hexanol; N-(3-aminopropyl) imidazole; N-(2-hydroxypropyl) imidazole; N-(2-hydroxyethyl) imidazole; N,N-bis(dimethylaminopropyl)-N-(3-aminopropyl)amine; N,N-bis(3-dimethylaminopropyl)-N-{3-[bis(2-hydroxypropyl)]propylamine}; N,N-bis(3-dimethylaminopropyl)-N-{3-[bis(2-hydroxyethyl)]propylamine}; N,N′-bis[bis-N″, N″-(3-dimethylaminopropyl)-N″-(3-aminopropyl)]urea; N,N-bis(3-dimethylaminopropyl)-N-(3-aminopropyl)]urea; N,N-bis(3-dimethylaminopropyl)-N-(bis(2-hydroxypropyl)-3-aminopropyl)]amine; N,N-bis(3-dimethylaminopropyl)-N—[N′,N′-bis(2-hydroxypropyl)-3-aminopropyl]amine; N, N-bis(3-dimethylaminopropyl)-N-[(2-hydroxypropyl)-3-aminopropyl]amine; 2-[N-(dimethylaminoethoxyethyl)-N-methylamino]ethanol; N, N-dimethylaminoethyl-N′-methyl-N′-ethanol; dimethylaminoethoxyethanol; N, N,N′-trimethyl-N′-3-aminopropyl-bis(aminoethyl) ether; or a combination thereof.

Item 7. The catalyst composition of any of items 1-6, wherein the catalyst composition is acid blocked with a carboxylic or sulfonic acid.

Item 8. The catalyst composition of item 7, wherein the composition is acid blocked with an acid selected from the group consisting of formic acid, acetic acid, propionic acid, butanoic acid, pentanoic acid, neopentanoic acid, hexanoic acid, 2-ethylhexyl carboxylic acid, neohexanoic acid, octanoic acid, neooctanoic acid, heptanoic acid, neoheptanoic acid, nonanoic acid, neononanoic acid, decanoic acid, neodecanoic acid, undecanoic acid, neoundecanoic acid, dodecanoic acid, neododecanoic acid, myristic acid, pentadecanoic acid, hexadecanoic acid, heptadecanoic acid, octadecanoic acid, benzoic acid, oxalic acid, malonic acid, succinic acid, glutaric acid, adipic acid, pimelic acid, suberic acid, azelaic acid, sebacic acid, glycolic acid, lactic acid, tartaric acid, citric acid, malic acid, and salicylic acid.

Item 9. The catalyst composition of any of items 1-8 further comprising a transition metal catalyst.

Item 10. The catalyst composition of claim 9 wherein the transition metal catalyst is an organotin compound, tin (II) carboxylate salt, bismuth (III) carboxylate salt, or combination thereof.

Item 11. The catalyst composition of any of items 1-10 further comprising catalytic materials selected from the group consisting of potassium formate, potassium acetate, potassium propionate, potassium butanoate, potassium pentanoate, potassium hexanoate, potassium heptanoate, potassium octoate, potassium 2-ethylhexanoate, potassium decanoate, potassium butyrate, potassium isobutyrate, potassium nonante, potassium stearate, sodium octoate, lithium stearate, sodium caprioate, lithium octoate, 2-hydroxypropyltrimethylammonium octoate solution, or any combination thereof.

Item 12. A polyurethane composition comprising the contact product of at least one active hydrogen-containing compound, at least one blowing agent, and the catalyst composition of any of items 1-3.

Item 13. The polyurethane composition of item 12, further comprising a tertiary amine having or not an isocyanate reactive group.

Item 14. The polyurethane composition of item 12 or 13, further comprising at least one additive selected from at least one cell stabilizer, at least one flame retardant, at least one chain extender, at least one epoxy resin, at least one acrylic resin, at least one filler, at least one pigment, or any combination thereof.

Item 15. A method for preparing a polyurethane foam comprising contacting at least one polyisocyanate with at least one active hydrogen-containing compound in the presence of at least one blowing agent and the catalyst composition defined in any of items 1-3.

Item 16. The method of item 15, wherein the catalyst composition is present in combination with a metal catalyst, a tertiary amine having or not an isocyanate reactive group, or a combination thereof.

### EXAMPLES

These Examples are provided to demonstrate certain embodiments of the invention and shall not limit the scope of the claims appended hereto.

### Example 1 (Inventive)

### This Example Describes the Synthesis of 2-[2-(dimethylamino)ethoxy]-N-methyl-acetamide (DMAEMAc)

In a fixed bed tubular reactor, equipped with a 10 cc quartz preheat bed, was charged with 8.8 g of a CuO/ZnO/Al₂O₃ catalyst sold under the name T-4581 material by Siid Chemie, with a typical composition of 61% CuO, 28% ZnO, and 10% Al₂O₃. The reactor was pressurized with nitrogen to 20.7 bar (300 psig), and then vented to ambient. The reactor pressure was maintained by means of a backpressure controller. The nitrogen purge was repeated two additional cycles, followed by three hydrogen purges. The reactor was then fed hydrogen at 500 scc/m and 20.7 bar (300 psig). The reactor was heated, at 1° C./minute with a resistance heater, to 250° C. and held at that temperature for 4 hr to reduce the catalyst. The hydrogen flow, metered via a mass flow controller, was adjusted to provide a 4/1 molar ratio of hydrogen/dimethylaminoethoxyethanol (DMAEE). DMAEE was fed to the reactor under pressure, via a constant flow syringe pump. MMA was co-fed to the reactor under pressure, via a constant flow syringe pump at an MMA/DMAEE molar ratio of 2/1. Effluent from the reactor was analyzed by GC to give approximately 12% of 2-[2-(dimethylamino)ethoxy]-N-methyl-acetamide (DMAEMAc) which was separated and purified by distillation.

### Example 2 (Inventive)

### This Example Describes the Synthesis of Crude 2-[2-(dimethylamino)ethoxy]-N-methyl-acetamide (Crude DMAEMAc)

A crude sample of DMAEMAc was made using the same procedure as described in Example 1. The sample was produced by distillation after removing N,N,N′-trimethyl-aminoethylether (TMAEE) as well as excess starting material (DMAEE) and small amounts of BDMAEE (bis-dimethylaminoethylether) to yield 25% of crude DMAEMAc. Crude DMAEMAc composition is mainly 50-60% DMAEMAc, 10-20% N-methyl-N,N-bis(dimethylaminoethoxyethyl)amine, 4-8% N,N-bis(dimethylaminoethoxyethyl)amine and about 4-6% 2-[2-(dimethylamino)ethoxy]-N-methyl-N-(dimethylamino ethoxyethyl)-acetamide.

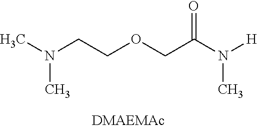

### Example 3 (Inventive)

### This Example Describes the Synthesis of Pure 2-[2-(dimethylamino)ethoxy]-N-methyl-acetamide (pure DMAEMAc)

A crude sample of DMAEMAc described in Example 2 was distilled off under nitrogen to give a clear liquid composed of DMAEMAc. The sample was produced by distillation after removing N-methyl-N,N-bis(dimethylaminoethoxyethyl)amine, N,N-bis(dimethylaminoethoxyethyl)amine, 2-[2-(dimethylamino)ethoxy]-N-methyl-N-(dimethylamino ethoxyethyl)-acetamide as well as other heavy impurities.

### Example 4 (Inventive)

### This Example Describes the Foam Rate of Rise Kinetics and Use Level Comparison for 2-[2-(dimethylamino)ethoxy]-N-methyl-acetamide (DMAEMAc)

Foaming performance can be evaluated by comparing the foam height versus time for standards and new amine catalyst. Foam height profile can be measured by automated rate of rise equipment, utilizing free-rise cup foam samples with a FOMAT sonar rate-of-rise device (hereafter referred to as a “ROR”). The FOMAT device comprises a sonar sensor that measures and records the height in millimeters (mm) of the rising foam sample versus time in seconds(s), directly after mixing all components of the formulation. The FOMAT standard software generates both height versus time plots and velocity versus time plots. These plots are useful for comparing the relative reactivity of different catalyst formulations. Flexible foam can be prepared by combining a total weight of about 300 g of the ingredients in Table 1 other than the isocyanate in a 32-oz (951 ml) paper cup. This premix formulation is then mixed for about 10 seconds at about 6,000 rpm using an overhead stirrer fitted with a 2-inch (5.1 cm) diameter stirring paddle. Sufficient toluene diisocyanate is then added to achieve the desired Isocyanate Index of about 100, and the formulation is mixed well for about another 6 seconds at about 6,000 rpm using the same stirrer. The cup is then placed under the FOMAT sensor. The start time for ROR measurement is automated for the FOMAT and begins directly after the end of the final mixing. Once the cup is placed under the ROR, the chemical mixture begins to polymerize. Since the walls of the cup restrict the expansion in all but the vertical direction, this expansion manifests itself in this experiment as an increase in height with passing time.

TABLE 1
Premix Components

| Component | PPHP |
|---|---|
| SPECFLEX ® NC 630¹ Polyol | 55 |
| SPECFLEX ® NC 700² Polyol | 45 |
| Water | 3.0 |
| DABCO ® DC6070³ | 0.60 |
| Low emissions | |
| silicon surfactant | |
| Catalyst⁴ | Varied |
| Diethanolamine | 0.70 |
| (crosslinker) | |
| Toluene | To provide NCO |
| diisocyanate | index = 100 |

¹High functionality capped polyether polyol of high molecular weight, functionality, and primary hydroxyl content with a base polyol molecular weight of about 5500, available from Dow Chemical Company, Midland, MI.
²Grafted polyether polyol containing copolymerized styrene and acrylonitrile, base polyol molecular weight about 4800, available from Dow Chemical Company, Midland, MI.
³Silicone surfactant is available from Evonik Corporation.
⁴The amine catalyst is available from Evonik Corporation

This increase in height can also be displayed as a rate of changing height (velocity) versus time. Useful comparisons can be made on the rate of the foaming reaction by recording the time required after mixing for the foam to reach a standard height (TOC=Top of the Cup), the maximum foam rise velocity, the time after mixing that was required to achieve the maximum velocity as well as the string gel time (SGT) which is the time at which the polymerizing mass is able to form polymer strings when touched with a wooden tongue suppressor.

TABLE 2
Foam Top of the Cup and String Gel Time in Seconds

| Run # | Gel Catalyst | pphp | TOC (sec) | SGT (sec) |
|---|---|---|---|---|
| 1 | DABCO ® NE1070¹ | 0.70 | 38 | 60 |
| 2 | Pure DMAEMAc | 0.95 | 40 | 60 |
| 3 | Crude DMAEMAc | 0.60 | 36 | 57 |

¹DABCO ®NE1070 is a mixture of N,N-dimethylaminopropylurea and bis(N,N-dimethylaminopropyl) urea in polyethylene glycol with average MW = 200 (PEG-200) catalyst commercially available from Air Products and Chemicals, Inc. Rate of rise data performed in all cases with 0.17 pphp of blowing amine catalyst N,N,N-trimethyl-N-3-aminopropyl-bis(aminoethyl) ether

### Example 5 (Inventive)

### This Example Compares the Physical Properties of Polyurethane Foam Made with Catalysts “Pure DMAEMAc”, “Crude DMAEMAc” and Standard Catalysts Used by the Polyurethane Industry

Foam pads were prepared by adding a tertiary amine catalyst to about 302 g of a premix (prepared as in Table 1) in a 32 oz (951 ml) paper cup. The formulation was mixed for about 10 seconds at about 6,000 RPM using an overhead stirrer fitted with a 2-inch (5.1 cm) diameter stirring paddle. The toluene diisocyanate was then added, and the formulation was mixed well for about another 6 seconds at about 6,000 RPM using the same stirrer, after which it was poured into a pre-heated mold at 70° C. and demolded after 4 minutes. The foam pads were removed from the mold, hand crushed, weighed and machine crushed at 75% pad thickness. Foam pads were stored under constant temperature and humidity condition for 48 hours before being cut and tested.

TABLE 3
Polyurethane TDI Flexible Molded Data

| Run# | Gel Catalyst | Gel Cat pphp | SG (sec) | Ext (sec) |
|---|---|---|---|---|
| 1 | DABCO ®33LV² | 0.30 | 58 | 42 |
| 2 | Pure DMAEMAc | 0.95 | 60 | 45 |
| 3 | Crude DMAEMAc | 0.60 | 55 | 40 |

¹Mold data performed in all cases with 0.10 pphp of blowing amine catalyst DABCO ®BL11 which is a 70% solution of bis(dimehtylaminoethyl) ether in dipropylene glycol. Dabco ®33LV² a 33% solution of TEDA (triethylenediamine) in dipropylene glycol.

TABLE 4
Physical Properties of TDI Polyurethane Flexible Molded Foam with 40 Kg/m³ Density and Index 100

| | | | | 40 kg/m³ Flex Molded, TDI Index 100 | | | | | |
|---|---|---|---|---|---|---|---|---|---|
| | | | | | | Pure | | Crude | |
| | | | | 33LV-BL11 | | DMAEMAc/BL11 | | DMAEMAc/BL11 | |
| Test | Sample | | Test | Av. 4 samples | | Av. 4 samples | | Av. 4 samples | |
| Description | Aging | Test Method ID | Units | Avg. | Std. | Avg. | Std. | Avg. | Std. |
| Core Densilty | Ambient | ASTM D3574-A | lbs/ft3 | 2.32 | 0.05 | 2.32 | 0.08 | 2.34 | 0.13 |
| Core Densilty | Ambient | ASTM D3574-A | (kg/m3) | 37.21 | 0.76 | 37.16 | 1.32 | 37.54 | 2.03 |
| Section Density | Ambient | ASTM D3574-A | lbs/ft3 | 2.65 | n/a | 2.62 | n/a | 2.63 | n/a |
| Section Density | Ambient | ASTM D3574-A | (kg/m3) | 42.52 | n/a | 41.98 | n/a | 42.13 | n/a |
| Air Flows | Ambient | ASTM D3574-G | SCFM | 1.89 | 0.24 | 2.07 | 0.15 | 1.82 | 0.14 |
| Air Flows | Ambient | ASTM D3574-G | L/M | 53.60 | 6.94 | 58.55 | 4.28 | 51.54 | 3.89 |
| ILD 25% | Ambient | ASTM D3574-B1 | lbsf | 43.39 | n/a | 36.58 | n/a | 36.83 | n/a |
| ILD 25% | Ambient | ASTM D3574-B1 | N | 193.02 | n/a | 162.73 | n/a | 163.82 | n/a |
| ILD 65% | Ambient | ASTM D3574-B1 | lbsf | 109.78 | n/a | 101.53 | n/a | 97.80 | n/a |
| ILD 65% | Ambient | ASTM D3574-B1 | N | 488.32 | n/a | 451.64 | n/a | 435.02 | n/a |
| ILD 25% Return | Ambient | ASTM D3574-B1 | Ibsf | 34.29 | n/a | 29.20 | n/a | 28.94 | n/a |
| ILD 25% Return | Ambient | ASTM D3574-B1 | N | 152.55 | n/a | 129.87 | n/a | 128.75 | n/a |
| Support Factor | Ambient | ASTM D3574-B1 | ratio | 2.53 | n/a | 2.78 | n/a | 2.66 | n/a |
| Resilience | Ambient | ASTM D3574-H | % | 54 | 1.0 | 53 | 0.6 | 52 | 0.0 |
| HALLS (70% Preflex) | Volkswagen | ISO-3386-1 | % | −23.65 | 1.05 | −19.66 | 0.97 | −30.35 | 0.67 |
| Tensile Strength | Ambient | DIN 53571 | psi | 22.63 | 1.29 | 22.07 | 1.33 | 22.43 | 1.67 |
| Tensile Strength | Ambient | DIN 53571 | kPa | 156.01 | 8.86 | 152.20 | 9.16 | 154.68 | 11.48 |
| Tensile Elongation | Ambient | DIN 53571 | % | 153.31 | 9.97 | 144.74 | 9.46 | 154.29 | 12.13 |
| HA Tensile Strength | Volkswagen | DIN 53571 | psi | 14.15 | 1.1 | 11.18 | 0.65 | 5.54 | 0.56 |
| HA Tensile Strength | Volkswagen | DIN 53571 | kPa | 97.59 | 7.85 | 77.07 | 4.45 | 38.16 | 3.85 |
| HA Elongation | Volkswagen | DIN 53571 | % | 167.33 | 3.46 | 136.99 | 8.22 | 82.33 | 5.08 |
| Tear Strength | Ambient | ASTM D3574-F | lbs | 2.03 | 0.12 | 2.13 | 0.31 | 1.98 | 0.11 |
| Tear Strength | Ambient | ASTM D3574-F | N | 9.01 | 0.52 | 9.46 | 1.37 | 8.81 | 0.47 |
| Tear Strength | Ambient | ASTM D3574-F | N/m | 354.96 | 20.59 | 372.54 | 53.94 | 347.11 | 18.45 |
| 50% Comp. Sets | 70° C. dry oven | ASTM D3574-D | % | 9.27 | 0.36 | 12.30 | 0.48 | 12.82 | 0.43 |
| 50% HA Comp. Sets | Volkswagen | ASTM D3574-D | % | 11.27 | 0.51 | 13.70 | 0.37 | 14.39 | 0.94 |

¹Mold data performed in all cases with 0.10 pphp of blowing amine catalyst DABCORBL 11 which is a 70% solution pf bis(dimehtylaminoethyl) ether in dipropylene glycol. Dabco ®33LV a 33% solution of TEDA (triethylenedimine) in dipropylene glycol. Volkswagen ageing procedure: Place samples to be tested in a dry oven at 90° C. for 24 hours for drying. Once dried, age samples for 200 hours 90° C and 100% relative humidity. Samples are then dried after.

Table 4 shows the ambient and humid aged physical properties of flexible molded polyurethane pads made with the standard reactive gelling/blowing amine catalysts Dabco®33LV/DABCO® BL11 and compared with the new gelling catalysts DMAEMAc (Example 3) and “Crude DMAEMAc” (Example 2) in both cases with blowing catalyst DABCO® NE300. Table 4 shows that the ambient physical properties were very similar providing foam pads with excellent physical properties. Table 4 also shows the physical properties after humid ageing using Volkswagen ageing procedure. The evaluation showed new gelling catalysts Pure-DMAEMAc and Crude-DMAEMAc performed similarly to a standard reactive catalyst DABCO®33LV, Foams made using the inventive catalyst have an overall better performance under humid ageing.

### Example 6 (Inventive)

### This Example Compares the Physical Properties of Polyurethane Foam Made with Catalysts “Pure-DMAEMAc”, “Crude-DMAEMAc” and Standard Catalysts Used by the Industry

Foam pads were prepared by adding a tertiary amine catalyst to about 302 g of a premix (prepared as in Table 1) in a 32 oz (951 ml) paper cup. The formulation was mixed for about 10 seconds at about 6,000 RPM using an overhead stirrer fitted with a 2-inch (5.1 cm) diameter stirring paddle. The toluene diisocyanate was then added, and the formulation was mixed well for about another 6 seconds at about 6,000 RPM using the same stirrer, after which it was poured into a pre-heated mold at 70° C. and demolded after 4 minutes. The foam pads were removed from the mold, hand crushed, weighed and machine crushed at 75% pad thickness. Foam pads were stored under constant temperature and humidity condition for 48 hours before being cut and tested.

TABLE 5
Polyurethane TDI Flexible Molded Data

| Run# | Gel Catalyst | Gel Cat pphp | SG (sec) | Ext (sec) |
|---|---|---|---|---|
| 1 | NE1070² | 0.70 | 58 | 40 |
| 2 | Pure-DMAEMAc | 0.95 | 61 | 45 |
| 3 | Crude-DMAEMAc | 0.60 | 56 | 37 |

¹Mold data performed in all cases with 0.17 pphp of blowing amine catalyst N,N,N′-trimethyl-N′-3-aminopropyl-bis(aminoethyl) ether;
²Dabco ®NE1070 a mixture of mono and bis-dimethylaminopropyl urea dissolved in polyethylene glycol-200

TABLE 6
Physical Properties of TDI Polyurethane Flexible Molded Foam with 40 Kg/m³ Density and Index 100

| | | | | 40 kg/m³ Flex Molded, TDI Index 100 | | | | | |
|---|---|---|---|---|---|---|---|---|---|
| | | | | NE1070-NE300 | | Pure DMAEMAc/NE300 | | Crude DMAEMAc/NE300 | |
| Test | Sample | | Test | Av. 4 samples | | Av. 4 samples | | Av. 4 samples | |
| Description | Aging | Test Method ID | Units | Avg. | Std. | Avg. | Std. | Avg. | Std. |
| Core Densilty | Ambient | ASTM D3574-A | lbs/ft3 | 2.39 | 0.05 | 2.41 | 0.06 | 2.30 | 0.01 |
| Core Densilty | Ambient | ASTM D3574-A | (kg/m3) | 38.26 | 0.80 | 38.56 | 1.01 | 36.88 | 0.17 |
| Section Density | Ambient | ASTM D3574-A | lbs/ft3 | 2.68 | n/a | 2.72 | n/a | 2.64 | n/a |
| Section Density | Ambient | ASTM D3574-A | (kg/m3) | 43.01 | n/a | 43.50 | n/a | 42.24 | n/a |
| Air Flows | Ambient | ASTM D3574-G | SCFM | 1.97 | 0.36 | 2.06 | 0.47 | 2.00 | 0.35 |
| Air Flows | Ambient | ASTM D3574-G | L/M | 55.86 | 10.19 | 58.27 | 13.43 | 56.57 | 9.84 |
| ILD 25% | Ambient | ASTM D3574-B1 | lbsf | 47.45 | n/a | 38.27 | n/a | 38.25 | n/a |
| ILD 25% | Ambient | ASTM D3574-B1 | N | 211.08 | n/a | 170.22 | n/a | 170.16 | n/a |
| ILD 65% | Ambient | ASTM D3574-B1 | lbsf | 106.18 | n/a | 104.09 | n/a | 94.75 | n/a |
| ILD 65% | Ambient | ASTM D3574-B1 | N | 472.33 | n/a | 463.04 | n/a | 421.48 | n/a |
| ILD 25% Return | Ambient | ASTM D3574-B1 | lbsf | 38.66 | n/a | 31.53 | n/a | 30.95 | n/a |
| ILD 25% Return | Ambient | ASTM D3574-B1 | N | 171.95 | n/a | 140.24 | n/a | 137.66 | n/a |
| Support Factor | Ambient | ASTM D3574-B1 | ratio | 2.24 | n/a | 2.72 | n/a | 2.48 | n/a |
| Resilience | Ambient | ASTM D3574-H | % | 58 | 0.6 | 58 | 0.6 | 57 | 0.6 |
| HALLS (70% Preflex) | Volkswagen | ISO-3386-1 | % | −44.63 | 2.16 | −27.45 | 1.70 | −25.52 | 2.25 |
| Tensile Strength | Ambient | DIN 53571 | psi | 18.90 | 0.15 | 21.41 | 1.92 | 21.95 | 2.05 |
| Tensile Strength | Ambient | DIN 53571 | kPa | 130.33 | 1.00 | 147.64 | 13.25 | 151.31 | 14.14 |
| Tensile Elongation | Ambient | DIN 53571 | % | 129.76 | 5.52 | 142.38 | 7.72 | 148.89 | 9.72 |
| HA Tensile Strength | Volkswagen | DIN 53571 | psi | 0.94 | 0.15 | 5.69 | 0.30 | 5.94 | 0.37 |
| HA Tensile Strength | Volkswagen | DIN 53571 | kPa | 6.47 | 1.03 | 39.24 | 2.09 | 40.98 | 2.55 |
| HA Elongation | Volkswagen | DIN 53571 | % | 42.34 | 2.70 | 73.66 | 6.41 | 69.99 | 7.58 |
| Tear Strength | Ambient | ASTM D3574-F | lbs | 1.92 | 0.08 | 1.78 | 0.12 | 1.69 | 0.12 |
| Tear Strength | Ambient | ASTM D3574-F | N | 8.55 | 0.35 | 7.91 | 0.55 | 7.53 | 0.51 |
| Tear Strength | Ambient | ASTM D3574-F | N/m | 337.07 | 13.98 | 311.54 | 21.57 | 296.57 | 20.25 |
| 50% Comp. Sets | 70° C. dry oven | ASTM D3574-D | % | 9.50 | 0.32 | 12.24 | 0.49 | 12.86 | 0.17 |
| 50% HA Comp. Sets | Volkswagen | ASTM D3574-D | % | 35.61 | 1.56 | 14.14 | 0.76 | 15.71 | 0.75 |

Mold data performed in all cases with 0.17 pphp of blowing amine catalyst N,N,N′-trimethyl-N′-(3-aminopropyl)-bis(aminoethyl) ether²; Volkswagen ageing procedure: Place samples to be tested in a dry oven at 90° C. for 24 hours for drying. Once dried, age samples for 200 hours 90° C. and 100% relative humidity. Samples are then dried after.

Table 6 shows the ambient and humid aged physical properties of flexible molded polyurethane pads made with the standard reactive gelling/blowing amine catalysts Dabco® NE1070/DABCO® NE300 and compared with the new gelling catalysts pure-DMAEMAc and crude-DMAEMAc in both cases with blowing catalyst DABCO® NE300. Table 6 shows that the ambient physical properties were very similar providing foam pads with excellent physical properties. Table 6 also shows the physical properties after humid ageing using Volkswagen ageing procedure. The evaluation showed new gelling catalysts pure-DMAEMAc and crude-DMAEMAc performed similarly to a standard reactive catalyst DABCO® NE1070, Foams made using the inventive catalyst have an overall better performance under humid ageing.

### Example 7 (Inventive)

### This Example Describes the Performance of DMAEMAc in a Typical Closed Cell Rigid Foam Formulation

The evaluation of the catalyst reactivity in a rigid polyurethane system was conducted using free-rise cup foam sample with a FOMAT sonar Rate-Of-Rise (ROR) device. The FOMAT standard software generates both height versus time plots and velocity versus time plots. These plots are useful for comparing the relative reactivity of different catalyst formulations. MDI polyurethane foam was prepared in a conventional hand mix manner. The polyurethane formulations in parts by weight:

| COMPONENT | WEIGHT % |
|---|---|
| Stepanpol ®PS-2352 | 50.00 |
| Pluraco ®SG-360 | 50.00 |
| TCPP | 10.00 |
| Water | 3.00 |
| Dabco DC-193 | 2.00 |
| Catalyst | Varied |
| N-Pentane | 5.00 |
| MDI Index | 136 |

¹Stepanpol ®PS2352 is a polyester polyol with a OH number of about 240 available from Stephan and Pluracol ®SG-360 is a polyol sucrose/glycerine initiated polyol with a functionality af 4 and a hydroxyl number of 350-375 available from BASF.
³Flame retardant: TCPP obtained from ICL-IP.
⁴Surfactant: DABCO ®DC193 obtained from Evonik Industries.
⁵Catalyst: varied

The table below shows the amount of catalyst needed for matching the string gel time in a standard rigid formulation when distilled (pure) DMAEMAc and non-distilled (crude) DMAEMAc are used as sole catalysts.

| | | | | | | Rise | Final |
|---|---|---|---|---|---|---|---|
| | | Cat 1 | CT | TOC | SGT | Time | Rise Ht |
| Run# | Catalyst 1 | pphp | (sec) | (sec) | (sec) | (sec) | (mm) |
| 1 | Pure DMAEMAc | 3.60 | 14-16 | 62.9 | 98 | 157.0 | 326.8 |
| 2 | Crude DMAEMAc | 1.70 | 10 | 56.2 | 98 | 146.8 | 324.6 |

Alternatively, as shown in the next table, DMAEMAc and non-distilled (crude) DMAEMAc can be used in a rigid formulation in combination with a blowing catalyst such as Polycat® 5 also commercially available from Evonik Corporation.

| | | | | | | | | Rise | Final |
|---|---|---|---|---|---|---|---|---|---|
| | | Cat 1 | | Cat2 | CT | TOC | SGT | Time | Rise Ht |
| Run# | Catalyst 1 | pphp | Catalyst 2 | pphp | (sec) | (sec) | (sec) | (sec) | (mm) |
| 3 | Pure DMAEMAc | 1.40 | Polycat 8 | 0.40 | 16 | 61 | 95 | 159 | 337 |
| 4 | Pure DMAEMAc | 1.30 | Polycat 5 | 0.30 | 10 | 53 | 94 | 143 | 331 |

The following tables show the physical properties of foam made with DMAEMAC:

ASTM D-6226% Closed Cell For Rigid Polurethanes (Formerly Under ASTM D-2856)
Quantachrome Penta-Pycnometer Project: 1-0972 Operator: SR Date: various

| | Sample | Volume | TRUE | Measured | Measured | % Closed | % Open | Measured | |
|---|---|---|---|---|---|---|---|---|---|
| | Instrument | 6 Spheres | Volume | Sample | Sample | Cell of | Cell of | Sample | Measured |
| | Reading | in Chamber | of Sample | Volume | Volume | Sample | Sample | Wgt. | Density |
| Sample I.D. | (cc) | (cc) | (cc) | (in3) | (cc) | (%) | (%) | (g) | (g/cc) |
| 1 | 21.6680 | 7.5075 | 14.1605 | 0.9761 | 15.9885 | 88.57 | 11.43 | 0.650 | 0.041 |
| 3.60p Pure | 21.7414 | 7.5075 | 14.2339 | 0.9841 | 16.1196 | 88.30 | 11.70 | 0.660 | 0.041 |
| DMAEMAc | 21.8637 | 7.5075 | 14.3562 | 0.9908 | 16.2293 | 88.46 | 11.54 | 0.680 | 0.042 |
| | 21.8206 | 7.5075 | 14.3131 | 0.9815 | 16.0770 | 89.03 | 10.97 | 0.680 | 0.042 |
| | 21.8372 | 7.5075 | 14.3297 | 0.9821 | 16.0868 | 89.08 | 10.92 | 0.640 | 0.040 |
| Average | 21.7862 | 7.5075 | 14.2787 | 0.9829 | 16.1002 | 88.6866 | 11.3134 | 0.6620 | 0.041 |
| Std. Deviation | 0.0803 | 0.0000 | 0.0803 | 0.0053 | 0.0869 | 0.3478 | 0.3478 | 0.0179 | 0.001 |
| 2 | 21.9746 | 7.5075 | 14.4671 | 1.0020 | 16.4128 | 88.15 | 11.85 | 0.660 | 0.040 |
| 1.70p Crude | 21.8800 | 7.5075 | 14.3725 | 0.9892 | 16.2031 | 88.70 | 11.30 | 0.660 | 0.041 |
| DMAEMAc | 21.7933 | 7.5075 | 14.2858 | 0.9899 | 16.2146 | 88.10 | 11.90 | 0.670 | 0.041 |
| 3 | 21.9907 | 7.5075 | 14.4832 | 0.9939 | 16.2801 | 88.96 | 11.04 | 0.680 | 0.042 |
| 1.40p Pure DMAEMAc | 22.0994 | 7.5075 | 14.5919 | 0.9860 | 16.1507 | 90.35 | 9.65 | 0.670 | 0.041 |
| 0.40p Polycat 8 | 22.4828 | 7.5075 | 14.9753 | 1.0080 | 16.5110 | 90.70 | 9.30 | 0.650 | 0.039 |
| | 22.2831 | 7.5075 | 14.7756 | 0.9950 | 16.2981 | 90.66 | 9.34 | 0.640 | 0.039 |
| | 22.1414 | 7.5075 | 14.6339 | 0.9868 | 16.1638 | 90.54 | 9.46 | 0.630 | 0.039 |
| | 22.1986 | 7.5075 | 14.6911 | 0.9980 | 16.3472 | 89.87 | 10.13 | 0.640 | 0.039 |
| | 22.0890 | 7.5075 | 14.5815 | 0.9998 | 16.3767 | 89.04 | 10.96 | 0.650 | 0.040 |
| Average | 22.2390 | 7.5075 | 14.7315 | 0.9975 | 16.3394 | 90.1598 | 9.8402 | 0.6420 | 0.039 |
| Std. Deviation | 0.1541 | 0.0000 | 0.1541 | 0.0077 | 0.1260 | 0.7112 | 0.7112 | 0.0084 | 0.000 |
| 4 | 22.2244 | 7.5075 | 14.7169 | 0.9990 | 16.3636 | 89.94 | 10.06 | 0.650 | 0.040 |
| 1.30p Pure DMAEMAc | 22.5168 | 7.5075 | 15.0093 | 1.0090 | 16.5274 | 90.81 | 9.19 | 0.650 | 0.039 |
| 0.30p Polycat 5 | 21.9475 | 7.5075 | 14.4400 | 0.9869 | 16.1654 | 89.33 | 10.67 | 0.660 | 0.041 |
| | 22.4339 | 7.5075 | 14.9264 | 1.0090 | 16.5274 | 90.31 | 9.69 | 0.670 | 0.041 |
| | 22.0625 | 7.5075 | 14.5550 | 0.9985 | 16.3554 | 88.99 | 11.01 | 0.670 | 0.041 |
| Average | 22.2370 | 7.5075 | 14.7295 | 1.0005 | 16.3879 | 89.8765 | 10.1235 | 0.6600 | 0.040 |
| Std. Deviation | 0.2406 | 0.0000 | 0.2406 | 0.0092 | 0.1501 | 0.7347 | 0.7347 | 0.0100 | 0.001 |

ASTM C 421 Friability of Rigid Polyurethane Foam
Standard Test Method for Tumbling Friability of Preformed
Block-Type Thermal Insulation

| | | First Cycle | Second Cycle | % Mass | % Mass |
|---|---|---|---|---|---|
| | Initial | Revolutions | Revolutions | Loss After | Loss After |
| | Weight | 600 (10 min) | 600 (+10 min) | First | Second |
| Sample Name | (grams) | weight, grams | weight, grams | Cycle | Cycle |
| 903-7: Pure DMAEMAc | 8.04 | 7.97 | 7.89 | 0.87 | 1.87 |
| 903-11: Crude DMAEMAc | 6.63 | 6.54 | 6.47 | 1.36 | 2.41 |
| 916-13: Polycat 8 | 6.25 | 6.11 | 6.02 | 2.24 | 3.68 |
| 916-14: Pure DMAEMAc/PC8 | 7.13 | 7.03 | 6.95 | 1.40 | 2.52 |
| 916-15: Pure DMAEMAc/PC5 | 8.29 | 8.22 | 8.17 | 0.84 | 1.45 |

Therefore, pure DMAEMAc and crude DMAEMAc are useful catalysts in the preparation of various types of polyurethane foams including rigid and flexible.

1. A catalyst composition comprising at least one compound represented by formula (I): 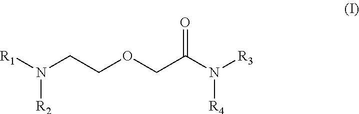
wherein:
R₁, R₂, and R₃, are each independently C₁-C₃ alkyl, or C₂-C₆ alkenyl linear or branched; and
R₄ is hydrogen, C₁-C₃ alkyl, or C₂-C₆ alkenyl linear or branched.

2. The catalyst composition of claim 1, wherein R₁, R₂, and R₃ are each independently methyl groups and R₄ is hydrogen.

3. The catalyst composition of claim 1, wherein the at least one compound represented by formula (I) is selected from the group consisting of 2-[2-(dimethylamino)ethoxy]-N-methyl-acetamide, 2-[2-(dimethylamino)ethoxy]-N,N-diethyl-acetamide, 2-[2-(dimethylamino)ethoxy]-N,N-dipropyl-acetamide, 2-[2-(dimethylamino)ethoxy]-N,N-dibutyl-acetamide, 2-[2-(dimethylamino)ethoxy]-N,N-dipentyl-acetamide, 2-[2-(dimethylamino)ethoxy]-N,N-dihexyl-acetamide, 2-[2-(dimethylamino)ethoxy]-N-methyl-N-ethyl-acetamide, 2-[2-(dimethylamino)ethoxy]-N-methyl-N-propyl-acetamide, 2-[2-(dimethylamino)ethoxy]-N-methyl-N-butyl-acetamide, 2-[2-(dimethylamino)ethoxy]-N-methyl-N-pentyl-acetamide, 2-[2-(dimethylamino)ethoxy]-N-methyl-N-hexyl-acetamide, 2-[2-(dimethylamino)ethoxy]-N-ethyl-acetamide, 2-[2-(dimethylamino)ethoxy]-N-propyl-acetamide, 2-[2-(dimethylamino)ethoxy]-N-butyl-acetamide, 2-[2-(dimethylamino)ethoxy]-N-pentyl-acetamide, 2-[2-(dimethylamino)ethoxy]-N-hexyl-acetamide, 2-[2-(diethylamino)ethoxy]-N,N-dimethyl-acetamide, 2-[2-(diethylamino)ethoxy]-N,N-diethyl-acetamide, 2-[2-(diethylamino)ethoxy]-N,N-dipropyl-acetamide, 2-[2-(diethylamino)ethoxy]-N,N-dibutyl-acetamide, 2-[2-(diethylamino)ethoxy]-N,N-dipentyl-acetamide, 2-[2-(diethylamino)ethoxy]-N,N-dihexyl-acetamide, 2-[2-(diethylamino)ethoxy]-N-methyl-N-ethyl-acetamide, 2-[2-(diethylamino)ethoxy]-N-methyl-N-propyl-acetamide, 2-[2-(diethylamino)ethoxy]-N-methyl-N-butyl-acetamide, 2-[2-(diethylamino)ethoxy]-N-methyl-N-pentyl-acetamide, 2-[2-(diethylamino)ethoxy]-N-methyl-N-hexyl-acetamide, 2-[2-(diethylamino)ethoxy]-N-methyl-acetamide, 2-[2-(diethylamino)ethoxy]-N-ethyl-acetamide, 2-[2-(diethylamino)ethoxy]-N-propyl-acetamide, 2-[2-(diethylamino)ethoxy]-N-butyl-acetamide, 2-[2-(diethylamino)ethoxy]-N-pentyl-acetamide, 2-[2-(diethylamino)ethoxy]-N-hexyl-acetamide, 2-[2-(N-methyl-N-ethyl-amino)ethoxy]-N,N-dimethyl-acetamide, 2-[2-(N-methyl-N-ethyl-amino)ethoxy]-N,N-diethyl-acetamide, 2-[2-(N-methyl-N-ethyl-amino)ethoxy]-N,N-dipropyl-acetamide, 2-[2-(N-methyl-N-ethyl-amino)ethoxy]-N,N-dibutyl-acetamide, 2-[2-(N-methyl-N-ethyl-amino)ethoxy]-N,N-dipentyl-acetamide, 2-[2-(N-methyl-N-ethyl-amino)ethoxy]-N,N-dihexyl-acetamide, 2-[2-(N-methyl-N-ethyl-amino)ethoxy]-N-methyl-N-ethyl-acetamide, 2-[2-(N-methyl-N-ethyl-amino)ethoxy]-N-methyl-N-propyl-acetamide, 2-[2-(N-methyl-N-ethyl-amino)ethoxy]-N-methyl-N-butyl-acetamide, 2-[2-(N-methyl-N-ethyl-amino)ethoxy]-N-methyl-N-pentyl-acetamide, 2-[2-(N-methyl-N-ethyl-amino)ethoxy]-N-methyl-N-hexyl-acetamide, 2-[2-(N-methyl-N-ethyl-amino)ethoxy]-N-methyl-acetamide, 2-[2-(N-methyl-N-ethyl-amino)ethoxy]-N-ethyl-acetamide, 2-[2-(N-methyl-N-ethyl-amino)ethoxy]-N-propyl-acetamide, 2-[2-(N-methyl-N-ethyl-amino)ethoxy]-N-butyl-acetamide, 2-[2-(N-methyl-N-ethyl-amino)ethoxy]-N-pentyl-acetamide, and 2-[2-(N-methyl-N-ethyl-amino)ethoxy]-N-hexyl-acetamide.

4. The catalyst composition of claim 1 further comprising a tertiary amine catalyst having or not an isocyanate reactive group.

5. The catalyst composition of claim 4, wherein the tertiary amine catalyst has at least one isocyanate reactive group comprising a primary hydroxyl group, a secondary hydroxyl group, a primary amine group, a secondary amine group, a urea group or an amide group.

6. The catalyst composition of claim 4 or 5, wherein the tertiary amine catalyst is selected from the group consisting of N, N-bis(3-dimethylaminopropyl)-N-isopropanolamine; N, N-dimethylaminoethyl-N′-methyl ethanolamine; N, N,N′-trimethylaminopropylethanolamine; N, N-dimethylethanolamine; N, N-diethylethanolamine; N, N-dimethyl-N′, N′-2-hydroxy (propyl)-1,3-propylenediamine; dimethylaminopropylamine; (N, N-dimethylaminoethoxy) ethanol; methyl-hydroxy-ethyl-piperazine; bis(N, N-dimethyl-3-aminopropyl)amine; N, N-dimethylaminopropyl urea; diethylaminopropyl urea; N,N′-bis(3-dimethylaminopropyl) urea; N,N′-bis(3-diethylaminopropyl) urea; bis(dimethylamino)-2-propanol; 6-dimethylamino-1-hexanol; N-(3-aminopropyl) imidazole; N-(2-hydroxypropyl) imidazole; N-(2-hydroxyethyl) imidazole; N,N-bis(dimethylaminopropyl)-N-(3-aminopropyl)amine; N, N-bis(3-dimethylaminopropyl)-N-{3-[bis(2-hydroxypropyl)]propylamine}; N,N-bis(3-dimethylaminopropyl)-N-{3-[bis(2-hydroxyethyl)]propylamine}; N,N′-bis[bis-N″,N″-(3-dimethylaminopropyl)-N″-(3-aminopropyl)]urea; N, N-bis(3-dimethylaminopropyl)-N-(3-aminopropyl)]urea; N, N-bis(3-dimethylaminopropyl)-N-(bis(2-hydroxypropyl)-3-aminopropyl)]amine; N, N-bis(3-dimethylaminopropyl)-N—[N′, N′-bis(2-hydroxypropyl)-3-aminopropyl]amine; N, N-bis(3-dimethylaminopropyl)-N-[(2-hydroxypropyl)-3-aminopropyl]amine; 2-[N-(dimethylaminoethoxyethyl)-N-methylamino]ethanol; N, N-dimethylaminoethyl-N′-methyl-N′-ethanol; dimethylaminoethoxyethanol; N, N,N′-trimethyl-N′-3-aminopropyl-bis(aminoethyl) ether; or a combination thereof.

7. The catalyst composition of claim 1, wherein the catalyst composition is acid blocked with a carboxylic or sulfonic acid.

8. The catalyst composition of claim 7, wherein the composition is acid blocked with an acid selected from the group consisting of formic acid, acetic acid, propionic acid, butanoic acid, pentanoic acid, neopentanoic acid, hexanoic acid, 2-ethylhexyl carboxylic acid, neohexanoic acid, octanoic acid, neooctanoic acid, heptanoic acid, neoheptanoic acid, nonanoic acid, neononanoic acid, decanoic acid, neodecanoic acid, undecanoic acid, neoundecanoic acid, dodecanoic acid, neododecanoic acid, myristic acid, pentadecanoic acid, hexadecanoic acid, heptadecanoic acid, octadecanoic acid, benzoic acid, oxalic acid, malonic acid, succinic acid, glutaric acid, adipic acid, pimelic acid, suberic acid, azelaic acid, sebacic acid, glycolic acid, lactic acid, tartaric acid, citric acid, malic acid, and salicylic acid.

9. The catalyst composition of claim 1 further comprising a transition metal catalyst.

10. The catalyst composition of claim 9 wherein the transition metal catalyst is an organotin compound, tin (II) carboxylate salt, bismuth (III) carboxylate salt, or combination thereof.

11. The catalyst composition of claim 1 further comprising catalytic materials selected from the group consisting of potassium formate, potassium acetate, potassium propionate, potassium butanoate, potassium pentanoate, potassium hexanoate, potassium heptanoate, potassium octoate, potassium 2-ethylhexanoate, potassium decanoate, potassium butyrate, potassium isobutyrate, potassium nonante, potassium stearate, sodium octoate, lithium stearate, sodium caprioate, lithium octoate, 2-hydroxypropyltrimethylammonium octoate solution, or any combination thereof.

12. A polyurethane composition comprising the contact product of at least one active hydrogen-containing compound, at least one blowing agent, and the catalyst composition of claim 1.

13. The polyurethane composition of claim 12, further comprising a tertiary amine having or not an isocyanate reactive group.

14. The polyurethane composition of claim 12, further comprising at least one additive selected from at least one cell stabilizer, at least one flame retardant, at least one chain extender, at least one epoxy resin, at least one acrylic resin, at least one filler, at least one pigment, or any combination thereof.

15. A method for preparing a polyurethane foam comprising contacting at least one polyisocyanate with at least one active hydrogen-containing compound in the presence of at least one blowing agent and the catalyst composition defined in claim 1.

16. The method of claim 15, wherein the catalyst composition is present in combination with a metal catalyst, a tertiary amine having or not an isocyanate reactive group, or a combination thereof.

17. The catalyst composition of claim 4 further comprising a transition metal catalyst.

18. The catalyst composition of claim 7 further comprising a transition metal catalyst.

19. The catalyst composition of claim 4 further comprising catalytic materials selected from the group consisting of potassium formate, potassium acetate, potassium propionate, potassium butanoate, potassium pentanoate, potassium hexanoate, potassium heptanoate, potassium octoate, potassium 2-ethylhexanoate, potassium decanoate, potassium butyrate, potassium isobutyrate, potassium nonante, potassium stearate, sodium octoate, lithium stearate, sodium caprioate, lithium octoate, 2-hydroxypropyltrimethylammonium octoate solution, or any combination thereof.

20. The polyurethane composition of claim 13, further comprising at least one additive selected from at least one cell stabilizer, at least one flame retardant, at least one chain extender, at least one epoxy resin, at least one acrylic resin, at least one filler, at least one pigment, or any combination thereof.
